(19) United States
(12) Patent Application Publication
(10) Pub. No.: US 2011/0190336 A1
(43) Pub. Date: Aug. 4, 2011

(54) AZABENZIMIDAZOLONES

(75) Inventors: Robert Zhiyong Luo, New City, NY (US); Mary-Margaret Zablocki, Fairfield, CT (US)

(73) Assignee: CARA THERAPEUTICS, INC., SHELTON, CT (US)

(21) Appl. No.: 13/062,794

(22) PCT Filed: Oct. 14, 2009
(86) PCT No.: PCT/US2009/060574

§ 371 Date: Mar. 8, 2011

Related U.S. Application Data
(60) Provisional application No. 61/105,912, filed on Oct. 16, 2008.
(60) Provisional application No. 61/180,448, filed on May 22, 2009.

(51) Int. Cl. A61K 31/437; C07D 471/04; A61P 25/00; A61P 29/00; A61P 37/08

(52) U.S. Cl. 514/303; 546/118

(57) ABSTRACT

Compounds are provided having the general structure of formula I:

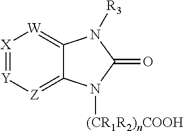

In formula I, one member of the group (W, X, Y and Z) is a nitrogen atom and the remaining three members of the group are each independently a carbon atom covalently bonded to a radical, R₄. The radicals, R₁, R₂, R₃ and R₄ are each defined herein, and n is an integer from 1 to 4. Also provided are stereoisomers, prodrugs, pharmaceutically acceptable salts, hydrates, salt hydrates, acid salt hydrates, and polymorphs of the compounds having the structure of formula I. The compounds bind the prostaglandin D2 receptor and are useful in the prophylaxis and treatment of prostaglandin D2-mediated diseases and conditions, including pain and inflammation, as well as asthma and allergic diseases and conditions.

Prostaglandin D2 (PGD2) is the major prostanoid metabolite produced by mast cells in response to allergen challenge, by the action of cyclooxygenases (COX-1 and COX-2) on arachidonic acid. PGD2 is elevated in a range of allergic conditions where it plays a prominent role due to its ability to induce extravasation of leukocytes and to act as a chemoattractant. The inflammatory effects of PGD2 are mediated through the interaction with two receptors, the D prostanoid receptor, DP1 and the Chemoattractant Receptor-homologous molecule expressed on T Helper Type 2 cells (CRTH2). [Pettipher, Br. J. Pharmacol. 153 Suppl 1:S191-9 (2008)]. CRTH2 is also known as DP2 (D prostanoid receptor 2) and alternatively as GPR44: G-protein coupled receptor 44 in the earlier literature. Antagonists of DP2 have been found to be useful in the prophylaxis and treatment of a range of prostaglandin D2-mediated diseases and conditions, including those associated with inflammation and allergies.

PGD2 is the most abundant prostaglandin in the central nervous system and has been implicated in a variety of neuronal functions including nociception. PGD2 acts not only by sensitizing peripheral terminals of primary afferent nociceptors, but also by augmenting the processing of pain signals at the spinal level. It has been shown that intrathecal administration of PGD2 induces hyperalgesic effects to noxious stimuli [Uda et al. Brain Res. 510 26-32 (1990)]. Paradoxically, PGD2 also inhibits the allodynic response to various chemical stimuli, and this response is restored in mice lacking the lipocalin-type PGD2 synthase [Eguchi et al., Proc. Natl. Acad. Sci. 96, 726-30 (1996)], the enzyme responsible for synthesis of PGD2 in the CNS.

### SUMMARY OF THE INVENTION

The present invention provides compounds having the general structure of formula I as shown below:

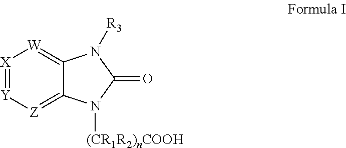

wherein one member of the group (W, X, Y and Z) is N and the remaining three members of the group (W, X, Y and Z) are each independently CR₄; the radicals R₁ and R₂ are each independently H or C₁-C₆ alkyl; and radical R₃ is

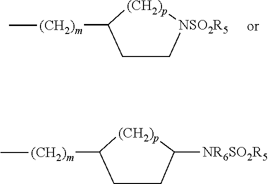

wherein each occurrence of the radical R₄ in formula I is independently chosen from hydrogen, hydroxyl, halo, nitro, cyano, C₁-C₆ alkylsulfonyl, C₁-C₆ alkyl, C₁-C₆ haloalkyl, C₁-C₆ alkoxy, C₁-C₆ haloalkoxy, C₁-C₆ aminoalkyl, C₁-C₆ alkylamino, C₁-C₆ alkylcarbamoyl, 6- to 10-membered aryl and 4- to 10-membered heterocyclyl.

The radical, R₅ is one of the following three alternatives: (i) 6- to 10-membered aryl optionally substituted with from 1 to 4 groups independently chosen from hydroxyl, halo, nitro, cyano, C₁-C₆ alkylsulfonyl, C₁-C₆ alkyl, C₁-C₆ haloalkyl, C₁-C₆ alkoxy, C₁-C₆ haloalkoxy, C₁-C₆ aminoalkyl, C₁-C₆ alkylamino, C₁-C₆ alkylcarbamoyl, phenyl and phenoxy; (ii) C₁-C₁₀ alkyl or C₃-C₈ cycloalkyl, each optionally substituted with from 1 to 4 groups independently chosen from hydroxyl, halo, nitro, cyano, C₁-C₆ alkyl, C₁-C₆ haloalkyl, C₁-C₆ alkoxy, C₁-C₆ haloalkoxy, C₁-C₆ alkylsulfonyl and phenyl; or (iii) 4- to 10-membered heterocyclyl optionally substituted with from 1 to 4 groups independently chosen from hydroxyl, halo, nitro, cyano, C₁-C₆ alkyl, C₁-C₆ haloalkyl, C₁-C₆ alkoxy, C₁-C₆ haloalkoxy and C₁-C₆ alkylsulfonyl. The radical, R₆ is hydrogen or C₁-C₄ alkyl.

In formula I, m is 0, 1, 2 or 3; n is 1, 2, 3 or 4; and p is 1, 2 or 3.

Also provided by the present invention are stereoisomers, prodrugs, pharmaceutically acceptable salts, hydrates, salt hydrates, acid salt hydrates, and polymorphs of the compounds having the structure of formula I. The compounds, stereoisomers, prodrugs, pharmaceutically acceptable salts, hydrates, salt hydrates, acid salt hydrates, and polymorphs of the compounds of the invention can be formulated with one or more pharmaceutically acceptable vehicles, diluents, carriers and/or excipients for clinical and veterinary uses.

Eicosanoids are biological lipid regulators of immune responses, including defenses against infection, ischemia, and injury, and play a role in initiating and perpetuating autoimmune and inflammatory conditions. The eicosanoid, PGD2 (prostaglandin D2) is known to act through at least two receptors, DP1 and DP2/CRTH2. The present invention provides compounds which are ligands of the CRTH2 (DP2) receptor. Moreover, the invention provides compounds and pharmaceutical compositions that include a compound having the structure of formula I, useful in the prophylaxis, inhibition and treatment of a range of diseases mediated by PGD2, including pain and inflammation, as well as asthma and allergic diseases and conditions.

The pain that is preventable or treatable by administration of a compound, or a pharmaceutical composition of a compound of the present invention, includes acute pain and chronic pain. Such pain addressable by administration of the compounds or pharmaceutical compositions of the invention includes, but is not limited to neuropathic pain, somatic pain, visceral pain and cutaneous pain.

Inflammatory and allergic disorders or conditions that can be prevented, treated, inhibited or managed by administration of the pharmaceutical compositions of the present invention include, but are not limited to, such disorders and conditions as rhinitis, asthma, chronic obstructive pulmonary disease, inflammatory bowel disease (IBD), allergic gestroenteropathy and contact dermatitis.

### DETAILED DESCRIPTION OF THE INVENTION

In the compounds having the general structure of formula I:

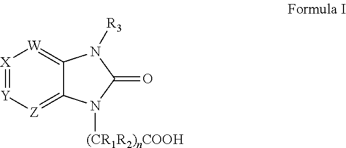

wherein one member of the group: W, X, Y and Z is a nitrogen atom; and the remaining three members of the group: W, X, Y and Z are each a carbon atom covalently bonded to an independently chosen radical, R₄.

The invention contemplates compounds of formula I having the structures Ia, Ib, Ic and Id as follows:

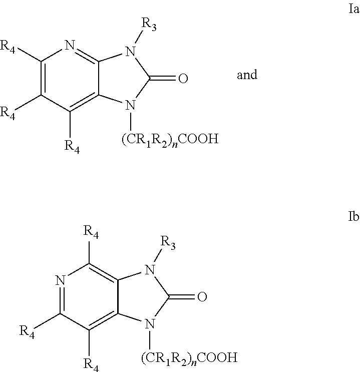

as well as

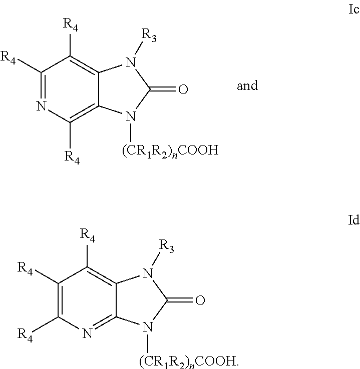

The radicals, R₁ and R₂ are each independently hydrogen or C₁-C₆ alkyl; and the radical R₃ has the structure:

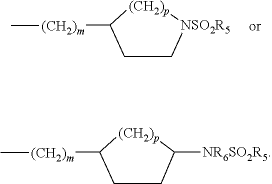

In formula I, each instance of R₄ is independently chosen from hydrogen, hydroxyl, halo, nitro, cyano, C₁-C₆ alkylsulfonyl, C₁-C₆ alkyl, C₁-C₆ haloalkyl, C₁-C₆ alkoxy, C₁-C₆ haloalkoxy, C₁-C₆ aminoalkyl, C₁-C₆ alkylamino, C₁-C₆ alkylcarbamoyl, 6- to 10-membered aryl and 4-, 5-6-7-8-9- and 10-membered heterocyclyl.

The radical, R₅ in formula I, is one of the following three alternatives:
(i) 6- to 10-membered aryl optionally substituted with from 1 to 4 groups independently chosen from hydroxyl, halo, nitro, cyano, C₁-C₆ alkylsulfonyl, C₁-C₆ alkyl, C₁-C₆ haloalkyl, C₁-C₆ alkoxy, C₁-C₆ haloalkoxy, C₁-C₆ aminoalkyl, C₁-C₆ alkylamino, C₁-C₆ alkyl-carbamoyl, heteroaryl, phenyl and phenoxy;
(ii) C₁-C₁₀ alkyl or C₃-Cₛ cycloalkyl, each optionally substituted with from 1 to 4 groups independently chosen from halo, hydroxyl, nitro, cyano, C₁-C₆ alkyl, C₁-C₆ haloalkyl, C₁-C₆ alkoxy, C₁-C₆ haloalkoxy, C₁-C₆ alkylsulfonyl and phenyl; and
(iii) 4- to 10-membered heterocyclyl optionally substituted with from 1 to 4 groups independently chosen from halo, hydroxyl, nitro, cyano, C₁-C₆ alkyl, C₁-C₆ haloalkyl, C₁-C₆ alkoxy, C₁-C₆ haloalkoxy and C₁-C₆ alkylsulfonyl.

The radical R₆ is hydrogen or C₁-C₄ alkyl.

In the compounds having the structure of formula I, m is zero or an integer from 1 to 3; n is an integer from 1 to 4; and p is an integer from 1 to 3.

The present invention also provides stereoisomers, prodrugs, pharmaceutically acceptable salts, hydrates, salt hydrates, acid salt hydrates, and polymorphs of the compounds having the structure of formula I. In one embodiment of the compounds of the present invention having the structure of formula I, the radicals R₁ and R₂ are each independently hydrogen, methyl or ethyl, and n is an integer from 1 to 3. In another embodiment, the compounds of the present invention have the structure of formula I, the radicals R₁ and R₂ are each hydrogen and n is equal to 1. In a third embodiment of the compounds having the structure of formula I, m is zero and p is equal to 2. In a fourth embodiment, the compounds having the structure of formula I, the integer, p is 1 or 2.

In another embodiment of the compounds of the present invention having the structure of formula I, at least one instance of the radical R₄ is hydrogen. In one aspect of this embodiment, at least two instances of R₄ are each hydrogen. In another aspect of this embodiment, three instances of R₄ are each hydrogen.

In still another embodiment of the compounds of the present invention having the structure of formula I, the radical R₅ is a 6- to 10-membered aryl optionally substituted with from 1 to 4 groups independently chosen from hydroxyl, halo, nitro, cyano, C₁-C₆ alkylsulfonyl, C₁-C₆ alkyl, C₁-C₆ haloalkyl, C₁-C₆ alkoxy, C₁-C₆ haloalkoxy, C₁-C₆ aminoalkyl, C₁-C₆ alkylamino, C₁-C₆ alkylcarbamoyl, heteroaryl, phenyl and phenoxy. In a further embodiment, the substituted aryl of R₅ is a 6-membered aryl. In another embodiment, the 6-membered aryl of R₅ is optionally substituted with from 1 to 4 groups independently chosen from halo, cyano, C₁-C₆ alkylsulfonyl, C₁-C₆ alkyl, C₁-C₆ haloalkyl, C₁-C₆ alkoxy.

In yet another embodiment of the compounds of the present invention having the structure of formula I, the radical R₅ is C₁-C₁₀ alkyl or C₃-Cₛ cycloalkyl, each optionally substituted with from 1 to 4 groups independently chosen from halo, hydroxyl, nitro, cyano, C₁-C₆ alkyl, C₁-C₆ haloalkyl, C₁-C₆ alkoxy, C₁-C₆ haloalkoxy, C₁-C₆ alkylsulfonyl and phenyl. In another embodiment, the radical R₅ is optionally substituted with from 1 to 4 groups independently chosen from halo, hydroxyl, nitro, cyano, C₁-C₆ alkoxy and C₁-C₆ haloalkoxy. Alternatively, the optional substituents of R₅ are chosen from halo and C₁-C₆ alkoxy.

In another embodiment of the compounds of the present invention having the structure of formula I, the radical R₅ is a 4- to 10-membered heterocyclyl optionally substituted with from 1 to 4 groups independently chosen from halo, hydroxyl, nitro, cyano, C₁-C₆ alkyl, C₁-C₆ haloalkyl, C₁-C₆ alkoxy, C₁-C₆ haloalkoxy and C₁-C₆ alkylsulfonyl. In another embodiment, the radical R₅ is an optionally substituted 5-, 6- or 7-membered heterocycle. In still another embodiment, the 5-, 6- or 7-membered heterocycle of R₅ is optionally substituted with from 1 to 4 groups independently chosen from halo, C₁-C₆ alkyl, C₁-C₆ haloalkyl, C₁-C₆ alkoxy and C₁-C₆ alkylsulfonyl.

The present invention also provides pharmaceutical compositions that include the compounds of formula I and pharmaceutically acceptable vehicle, diluent or excipient.

In another embodiment the present invention provides a method of prophylaxis or treatment of a disease or condition mediated by or addressable by DP2, wherein the method includes administering an effective amount of a compound according of formula Ito a subject in need thereof. The disease or condition mediated by or addressable by DP2 can be any disease or condition mediated or addressable by DP2, including but not limited to pain, including acute and chronic pain, inflammation and inflammatory pain, and allergic diseases and conditions such as allergic asthma, allergic rhinitis, atopic dermatitis, psoriasis, contact hypersensitivity, allergic conjunctivitis, allergic bronchitis and food allergies, as well as chronic obstructive pulmonary disease (COPD), and immune diseases and conditions such as rheumatoid arthritis, osteoarthritis, inflammatory bowel disease (IBS) and irritable bowel syndrome (IBD), and autoimmune diseases such as systemic lupus erythematosus, psoriasis, acne and multiple sclerosis.

General Methods

All reactions involving moisture sensitive compounds were carried out under an anhydrous nitrogen or argon atmosphere. All reagents were purchased from commercial sources and used without further purification. Unless otherwise noted, the starting materials used in the examples were obtained from readily available commercial sources or synthesized by standard methods known to those skilled in the art of organic synthesis.

Reactions performed under microwave irradiation conditions were carried out in a Biotage Initiator® 60 microwave system (Charlottesville, Va.; model# 10986-22V) with a 300 watt magnetron. Normal phase chromatography and reverse phase chromatography was performed on an ISCO CombiFlash® Companion® or CombiFlash® Companion/TS® system (Teledyne Isco, Inc., Lincoln, Nebr.). Preparative LC-MS was performed with a Waters (Waters Corporation, Milford, Mass.) HPLC-MS system equipped with a 2767 Sample Manager, 2545 Binary Gradient Module, SFO System Fluidics Organizer, 2996 Photodiode Array Detector and 3100 Mass Detector. Data was collected across a range of wavelengths from 220 nm to 280 nm and in positive electrospray-chemical ionization mode. The HPLC column used was a Waters XBridge C18 5 μm 4.6×150 mm. Spectra were scanned from 100-1400 amu. The eluents were A: water with 0.1% formic acid and B: acetonitrile with 0.1% formic acid. Gradient elution from 5% B to 95% B over 10 minutes was used with an initial hold of 1.2 minutes and final hold at 95% B of 1.0 minutes at a flow rate of 20 mL/min.

Compounds were characterized by their LC-MS-Electrospray/chemical ionization mass spectra (LC ESCI-MS) on one of the following systems:

1) Waters HPLC-MS system (Waters Corp., Milford, Mass.) equipped with a 2767 Sample Manager, 2545 Binary Gradient Module, SFO System Fluidics Organizer, 2996 Photodiode Array Detector and 3100 Mass Detector. Data were collected across a range of wavelengths from 220 nm to 280 nm in positive ESCI mode. Spectra were scanned from 100-1400 atomic mass units (amu). The HPLC column was a Waters XBridge C18 3.5 μm 4.6×30 mm; eluents were A: water with 0.1% formic acid and B: acetonitrile with 0.1% formic acid. Gradient elution was from 5% B to 95% B over 2.3 minutes with an initial hold of 0.2 minutes and a final hold at 95% B of 0.5 minutes. Total run time was 4 minutes.

2) Waters (Waters Corporation, Milford, Mass.) HPLC-MS system equipped with an Acquity Sample Manager, Acquity Binary Solvent Manager, Acquity Photodiode Array Detector, Acquity Evaporative Light Scattering Detector and SQ Detector. Data were collected at 220 nm and 254 nm and in positive electrospray-chemical ionization mode. The HPLC column used was a Waters Acquity HPLC BEH C18 1.7 μm 2.1×50 mm. Spectra were scanned from 100-1400 amu. The eluents were A: water with 0.1% formic acid and B: acetonitrile with 0.1% formic acid. Gradient elution from 5% B to 95% B over 0.8 minutes was used with a final hold at 95% B of 0.2 minutes at a flow rate of 0.8 milliliters per minute. Total run time was 1.5 minutes.

Nuclear magnetic resonance spectra were recorded using a Bruker Avance III (400 MHz shielded) spectrometer equipped with a Gradient Multinuclear Broadband Fluorine Observe (BBFO) probe. Spectra were acquired in the indicated solvent. Chemical shifts (δ) are given in ppm (parts per million upfield or downfield from TMS defined as 0 ppm). Coupling constants J are in hertz (Hz). Peak shapes in the NMR spectra are indicated by symbols ‘q’ (quartet), ‘t’ (triplet), ‘d’ (doublet), ‘s’ (singlet), ‘br s’ (broad singlet), ‘br’ (broad) ‘m’ (multiplet) and ‘br d’ (broad doublet).

Abbreviations Used Herein:

As used herein AcOH means acetic acid; CDI means carbonyl diimidazole; DCM means dichloromethane; DMF means dimethylformamide; DIEA means N,N-diiso-propylethylamine; DMAP means 4-dimethylaminopyridine; ESI means electron spray ionization; EtOAc means ethyl acetate; HCl means hydrochloric acid; ¹H-NMR means proton nuclear magnetic resonance; LC-MS means liquid chromatography-mass spectro-metry; LiOH means lithium hydroxide; MeOH means methanol; NaHCO₃ means sodium bicarbonate; Na₂SO₄ means sodium sulphate; TFA means trifluoroacetic acid; THF means tetrahydrofuran; and TLC means thin layer chromatography.

Synthetic Schemes 1-6

Compounds of the present invention can be prepared according to the non-limiting synthetic methods detailed in the following general schemes. Two series of compounds, 7a (wherein W═N; and each of X, Y and Z is a CR₄ radical) and 7b (wherein Y═N; and each of W, X and Z is a CR₄ radical), can be synthesized according to Scheme 1.

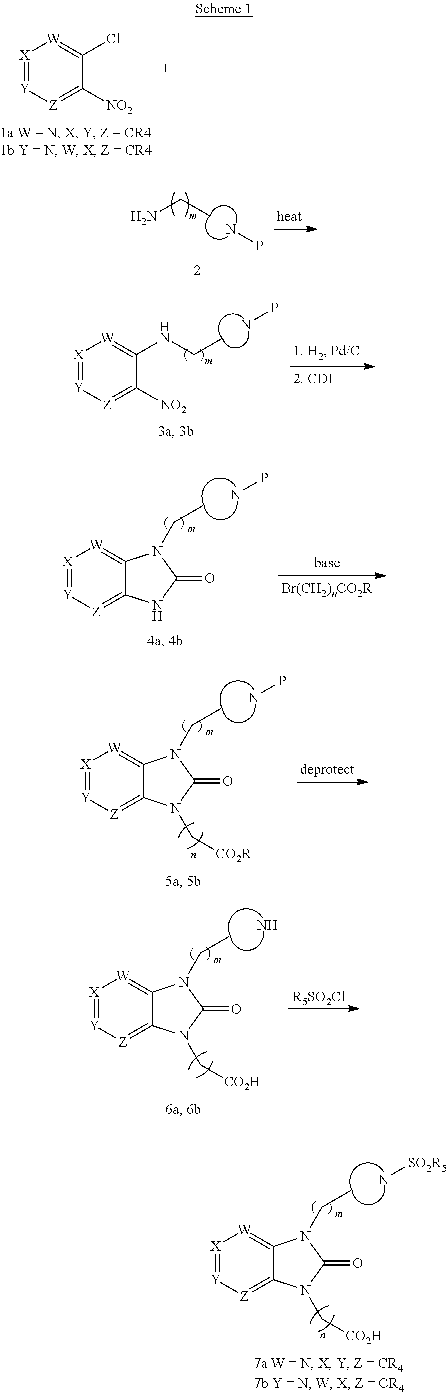

Commercially available 2-chloro-3-nitropyridine, 1a or 4-chloro-3-nitropyridine, 1b is reacted with a mono-protected diamine 2 under conventional or microwave heating conditions to give intermediate 3a or 3b, respectively. Reduction of the nitro group with hydrogen catalyzed by transition metals, such as palladium, is followed by ring closure with CDI, phosgene or triphosgene to provide intermediates 4a and 4b.

Deprotonation of intermediates 4a and 4b with a strong base such as sodium hydride followed by alkylation with commercially available bromoester derivatives such as tert-butyl bromoacetate, tert-butyl 3-bromopropionate, tert-butyl 4-bromobutyrate or ethyl 5-bromovalerate gives intermediate 5a and 5b. The nitrogen protecting group and alkyl ester can be deprotected under standard procedures as described in Greene's Protective Groups in Organic Synthesis (4th edition, Wiley, 2006) to give intermediate 6a and 6b, which in turn can be reacted with sulfonylchlorides to give two series of compounds, 7a and 7b, respectively.

The pyridine derivatives, 1a and 1b can also be used to synthesize two regioisomeric series of compounds disclosed in this application according to Scheme 2. Displacement of the chlorine atom of 1a or 1b with amino ester derivatives such as glycine ethyl ester, β-alanine ethyl ester, ethyl 4-aminobutyrate or ethyl 5-aminovalerate under conventional or microwave heating condition provides intermediates, 8a and 8b, respectively. Reduction of the nitro group with hydrogen catalyzed by transition metals such as palladium followed by ring closure with CDI, phosgene or triphosgene yields intermediates, 9a and 9b. Alkylation of the urea nitrogen of 9a and 9b can be accomplished by a Mitsunobu reaction with commercially available alcohols 10. Hydrolysis of the ester under basic conditions such as LiOH/MeOH/water followed by deprotection of the nitrogen protecting group gives intermediates 12a and 12b, which can in turn be reacted with sulfonylchlorides to give two series of compounds, 13a and 13b, respectively.

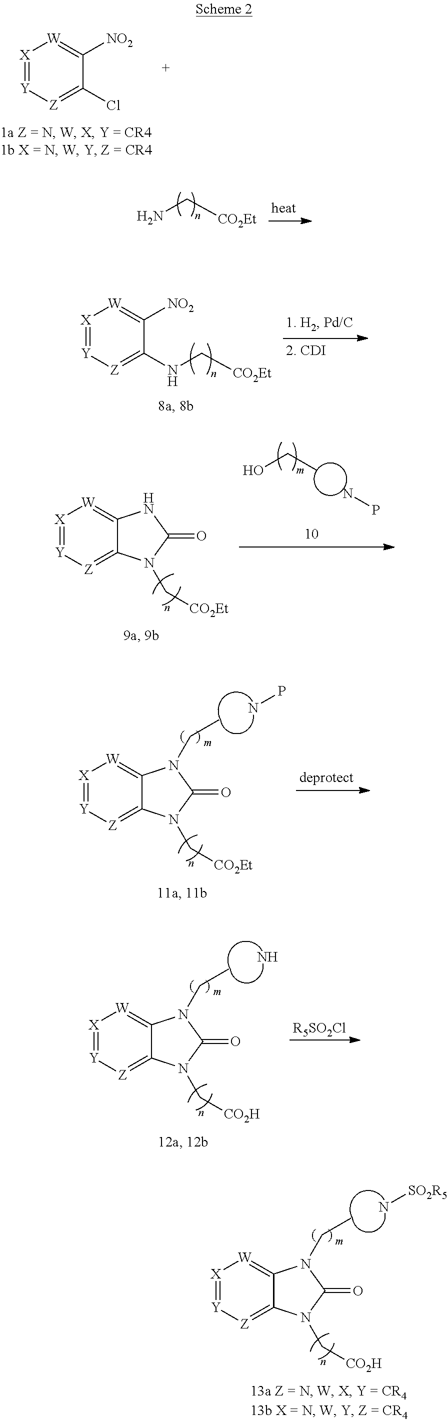

Schemes 3 to 6 illustrate the synthesis of compounds with a substituent on the pyridine ring. Following the synthetic sequences described in Scheme 1, intermediate 15 can be prepared from 2-chloro-3-nitro-5-bromopyridine 14 (Scheme 3).

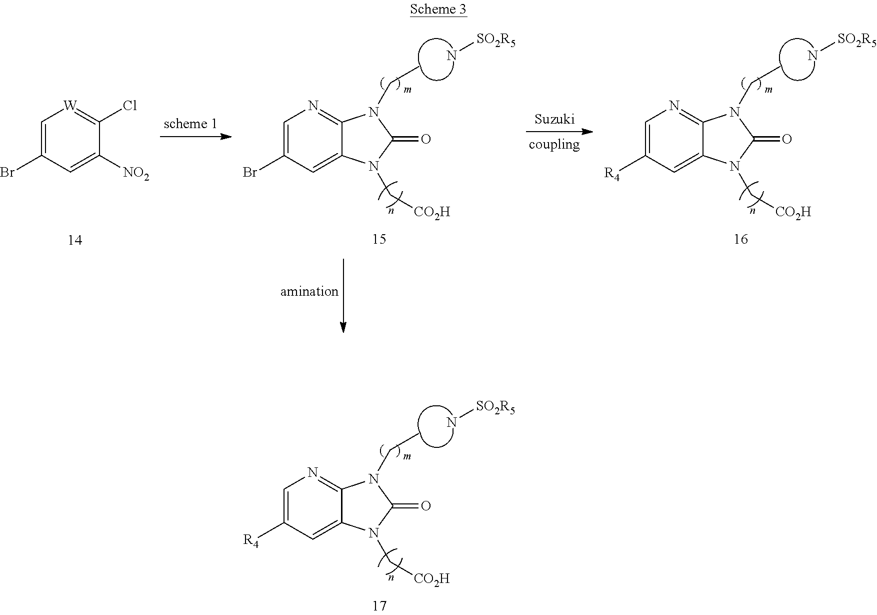

Suzuki coupling between intermediate 15 and aromatic or heterocyclyl aromatic (heteroaromatic) boronic acids provides compounds 16 with R₄ being aryl or heteroaryl. Alternatively, intermediate 15 can undergo transition metal catalyzed coupling reactions with amines to give compounds 17 with R₄ being alkylamino or dialkylamino groups.

Following the synthetic sequences described in Scheme 2, intermediate 18 can be prepared from 2-chloro-3-nitro-5-bromopyridine 14 (Scheme 4).

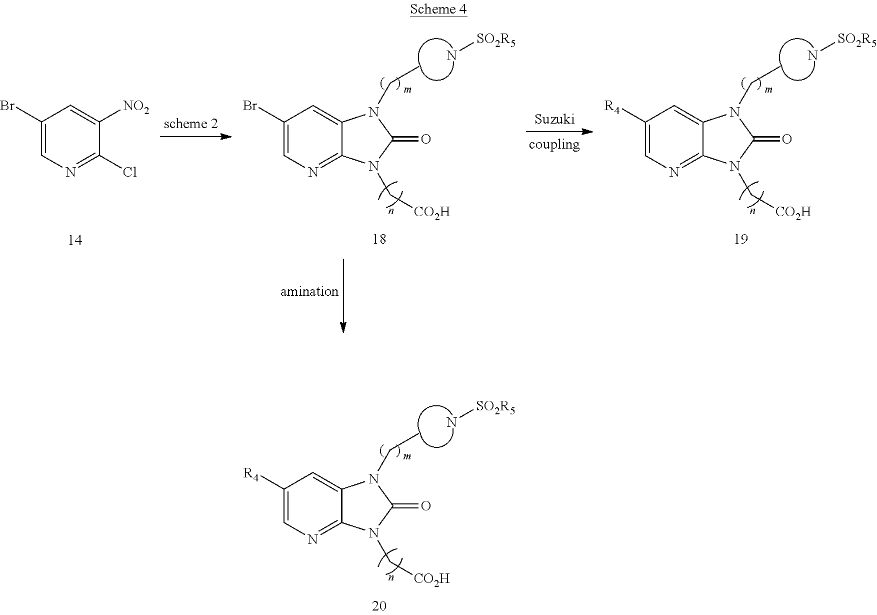

Suzuki coupling between intermediate 18 and aromatic or heterocyclyl aromatic boronic acids provides compounds 19 with R₄ being aryl or heteroaryl. Alternatively, intermediate 18 can undergo transition metal catalyzed coupling reactions with amines to give compounds 20 with R₄ being an alkylamino or a dialkylamino group.

Following the synthetic sequences described in Scheme 1, intermediate 22 can be prepared from 3-bromo-4-chloro-5-nitropyridine 21 (Scheme 5). Suzuki coupling between intermediate 22 and aromatic or heterocyclyl aromatic boronic acids provides compounds 23 with R₄ being aryl or heteroaryl. Alternatively, intermediate 22 can undergo transition metal catalyzed coupling reactions with amines to give compounds 24 with R₄ being an alkylamino or a dialkylamino group.

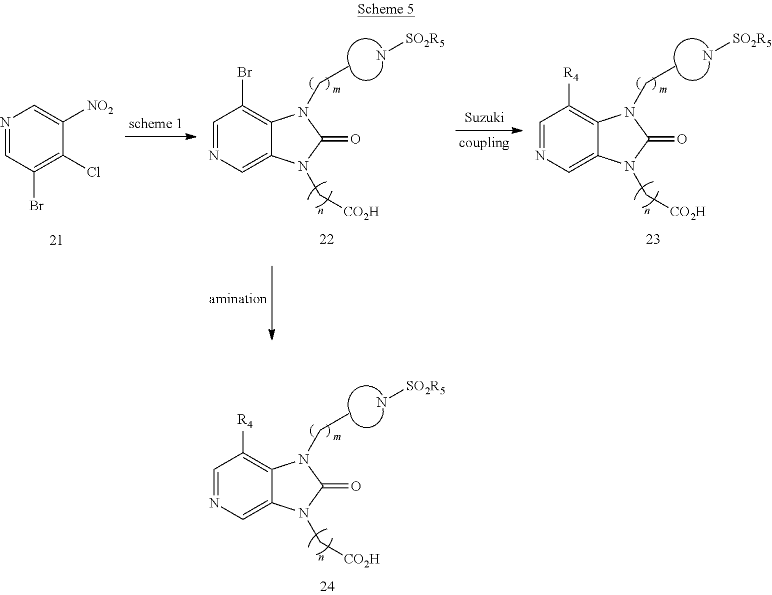

Following the synthetic sequences described in Scheme 2, intermediate 25 can be prepared from 3-bromo-4-chloro-5-nitropyridine 21 (Scheme 6).

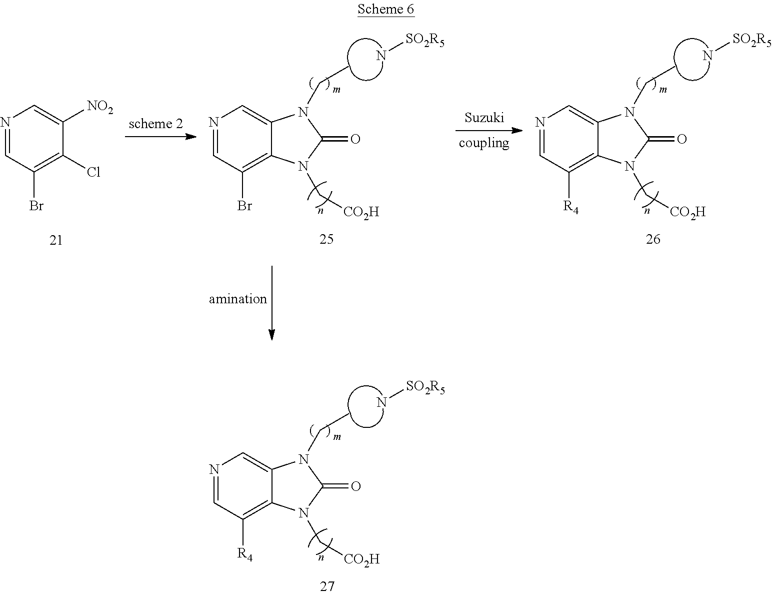

Suzuki coupling between intermediate 25 and aromatic or heterocyclyl aromatic boronic acids provides compounds 26 with R₄ being an aryl or heteroaryl group. Alternatively, intermediate 25 can undergo transition metal catalyzed coupling reactions with amines to give compounds 27 with R₄ being an alkylamino or a dialkylamino group.

### EXAMPLES

### Intermediate 28: Preparation of 2-(2-oxo-3-(piperidin-4-yl)-2,3-dihydro-1H-imidazo[4,5-b]pyridin-1-yl)acetic acid (28)

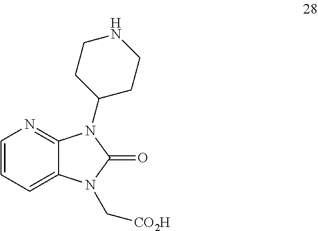

### Step 1: Preparation of tert-butyl 4-(3-nitropyridin-2-ylamino)piperidine-1-carboxylate

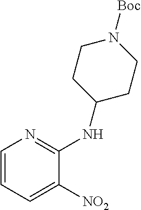

A mixture of 2-chloro-3-nitropyridine (1.59 g), tert-butyl 4-aminopiperidine-1-carboxylate (2.20 g) and DIEA (3.5 mL) in acetonitrile was heated by microwave irradiation at 135° C. for 30 min. After evaporation of acetonitrile, the residue was extracted between EtOAc and aqueous NaHCO₃. The organic phase was dried over sodium sulfate and evaporated to give tert-butyl 4-(3-nitropyridin-2-ylamino) piperidine-1-carboxylate as a solid (yield: 2.9 g), which is used in Step 2 without further purification. LC-MS (+ESI) m/z=323 [M+H]⁺.

### Step 2: Preparation of tert-butyl 4-(3-aminopyridin-2-ylamino)piperidine-1-carboxylate

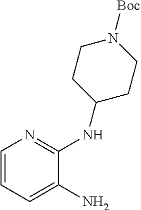

A mixture of tert-butyl 4-(3-nitropyridin-2-ylamino)piperidine-1-carboxylate (2.9 g), palladium on carbon (10%, 1.07 g) in methanol was hydrogenated under hydrogen (60 psi) in a Parr shaker for 1 hour. The catalyst was removed by filtration through celite. Evaporation of the filtrate under vacuum give tert-butyl 4-(3-amino-pyridin-2-ylamino)piperidine-1-carboxylate as a solid (yield: 2.6 g), which is used in Step 3 without further purification. LC-MS (+ESI) m/z=293 [M+H]⁺.

### Step 3: Preparation of tert-butyl 4-(2-oxo-1H-imidazo[4,5-b]pyridin-3(2H)-yl)piperidine-1-carboxylate

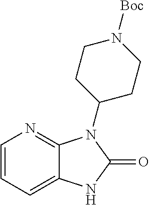

Triphosgene (2.98 g) was added portionwise to a solution of tert-butyl 4-(3-aminopyridin-2-ylamino)piperidine-1-carboxylate (2.6 g) and DIEA (3.5 mL) in DCM (50 mL) at 0° C. After stirring at 0° C. for 30 min, aqueous NaHCO₃ was added and stirred at room temperature for 15 min. The organic phase was separated and dried over anhydrous Na₂SO₄. After evaporation of DCM, the residue was purified by column chromatography with 50% to 80% EtOAc/hex to give tert-butyl 4-(2-oxo-1H-imidazo[4,5-b]pyridin-3(2H)-yl)piperidine-1-carboxylate as a solid (Yield: 2.27 g). LC-MS (+ESI) m/z=318 [M+H]⁺.

### Step 4: Preparation of tert-butyl 4-(1-(2-tert-butoxy-2-oxoethyl)-2-oxo-1H-imidazo[4,5-b]pyridin-3(2H)-yl)piperidine-1-carboxylate

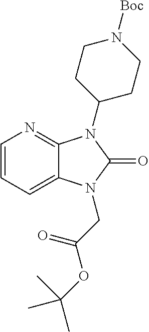

To a solution of tert-butyl 4-(2-oxo-1H-imidazo[4,5-b]pyridin-3(2H)-yl)piperidine-1-carboxylate (2.27 g) in anhydrous THF (50 mL) was added sodium hydride (0.40 g, 60% dispersion) at 0° C. After stirring at 0° C. for 10 min, tert-butyl bromoacetate (1.50 mL) was added and the mixture was stirred at 0° C. for 1 hour. The reaction was quenched with aqueous ammonium chloride and extracted with EtOAc. The organic phase was dried over sodium sulfate and evaporated to dryness. The residue was purified by column chromatography with 20% to 50% EtOAc/hex to give tert-butyl 4-(1-(2-tert-butoxy-2-oxoethyl)-2-oxo-1H-imidazo[4,5-b]pyridin-3(2H)-yl)piperidine-1-carboxylate as a solid (yield: 2.58 g). ¹H NMR (CDCl₃) δ 8.02 (dd, J=1.4, 5.2 Hz, 1H), 7.08 (dd, J=1.4, 7.7 Hz, 1H), 6.98 (dd, J=5.2, 7.8 Hz, 1 h), 4.54-4.57 (m, 1H), 4.52 (s, 2H), 4.34 (br, 2H), 2.84 (br, 2H), 2.59-2.70 (m, 2H), 1.77-1.83 (m, 2H), 1.50 (s, 9H), 1.48 (s, 9H). LC-MS (+ESI) m/z=433 [M+H]⁺.

### Step 5: Preparation of 2-(2-oxo-3-(piperidin-4-yl)-2,3-dihydro-1H-imidazo[4,5-b]pyridin-1-yl)acetic acid (28)

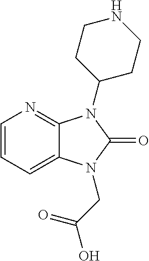

A solution of tert-butyl 4-(1-(2-tert-butoxy-2-oxoethyl)-2-oxo-1H-imidazo[4,5-b]pyridin-3(2H)-yl)piperidine-1-carboxylate (2.58 g) was stirred in TFA/DCM (3:1) at room temperature for 2 hours. After evaporation of TFA and DCM, the residue was azeotroped with toluene twice to give the TFA salt of 2-(2-oxo-3-(piperidin-4-yl)-2,3-dihydro-1H-imidazo[4,5-b]pyridin-1-yl)acetic acid as an oil. LC-MS (+ESI) m/z=277 [M+H]⁺.

### Intermediate 29: Preparation of 2-(6-fluoro-2-oxo-3-(piperidin-4-yl)-2,3-dihydro-1H-imidazo[4,5-b]pyridin-1-yl)acetic acid (29)

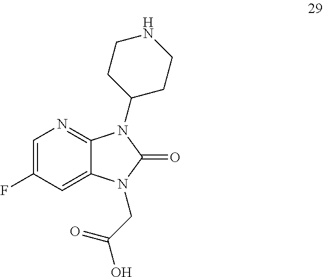

Intermediate 29 was prepared according to the procedure described for the synthesis of intermediate 28 by replacing 2-chloro-3-nitropyridine with 2-chloro-3-nitro-5-fluoropyridine.

### Example 1

### Preparation of 2-(3-(1-(4-fluoro-2-methylphenylsulfonyl)piperidin-4-yl)-2-oxo-2,3-dihydro-1H-imidazo[4,5-b]pyridin-1-yl)acetic acid (30)

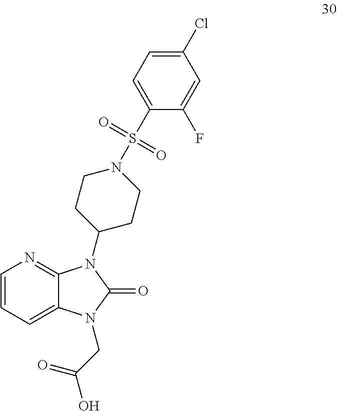

To a solution of 2-(2-oxo-3-(piperidin-4-yl)-2,3-dihydro-1H-imidazo[4,5-b]pyridin-1-yl)acetic acid 28 (25 mg) in THF/water (1:1) was added DIEA (25 μL) followed by 2-fluoro-4-chlorobenzenesulfonyl chloride (30 μL). After stirring at room temperature for 2 hours, the reaction mixture was concentrated and diluted with methanol (0.5 mL). The reaction mixture was purified by prep LC-MS with a 14 mins gradient from 5% MeCN/water to 95% MeCN/water. Pure fractions were evaporated to give 2-(3-(1-(4-chloro-2-fluorophenylsulfonyl)piperidin-4-yl)-2-oxo-2,3-dihydro-1H-imidazo[4,5-b]pyridin-1-yl)acetic acid 30 (yield: 3.2 mg). LC-MS (+ESI) m/z=469 [M+H]⁺.

Compounds 31-127 (shown in Table 1) were prepared according to the procedure described for the synthesis of compound 30 by replacing 2-fluoro-4-chlorobenzene-sulfonyl chloride with the corresponding sulfonyl chlorides. For example, compound 34 was prepared by a reaction between intermediate 28 with cyclohexylsulfonyl chloride. Compounds 71-74 were prepared according to the procedure described for the synthesis of compound 30 by replacing intermediate 28 with intermediate 29 and sulfonylating with 2-fluoro-4-chlorobenzenesulfonyl chloride, quinoline-8-sulfonyl chloride, 4-isopropyl-oxybenzene-sulfonyl chloride or 2-methylsulfonylbenzenesulfonyl chloride, respectively. Table 1 shows the structures of compounds 31-127.

TABLE 1
Compound Structures

| Cmpd # | Structure | Chemical Name | m/z |
|---|---|---|---|
| 31 | 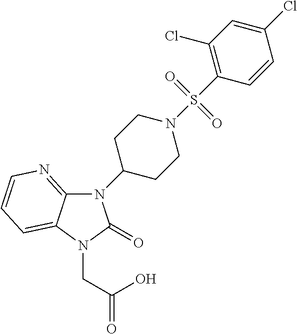 | 2-(3-(1-(2,4-dichlorophenyl- sulfonyl)piperidin-4-yl)-2-oxo-2,3- dihydro-1H-imidazo[4,5-b]pyridin-1- yl)acetic acid | 484.9 |
| 32 | 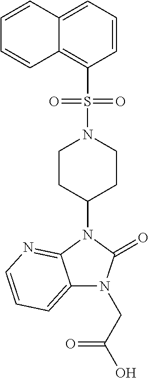 | 2-(3-(1-(naphthalen-1-ylsulfonyl) piperidin-4-yl)-2-oxo-2,3-dihydro-1H- imidazo[4,5-b]pyridin-1-yl)acetic acid | 467.0 |
| 33 | 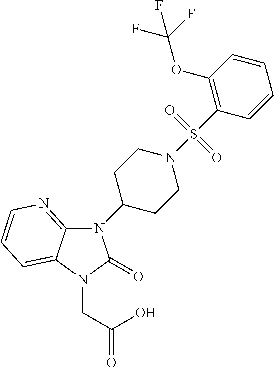 | 2-(2-oxo-3-(1-(2-(trifluoromethoxy) phenylsulfonyl)piperidin-4-yl)-2,3- dihydro-1H-imidazo[4,5-b]pyridin-1- yl)acetic acid | 501.0 |
| 34 | 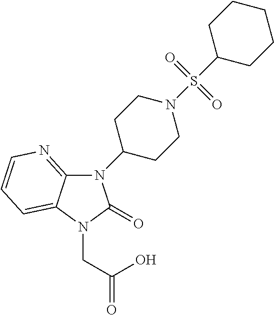 | 2-(3-(1-(cyclohexylsulfonyl)piperidin- 4-yl)-2-oxo-2,3-dihydro-1H-imidazo [4,5-b]pyridin-1-yl)acetic acid | 423.1 |
| 35 | 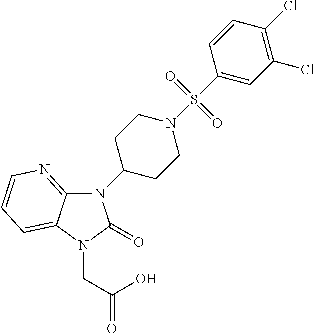 | 2-(3-(1-(3,4-dichlorophenyl- sulfonyl)piperidin-4-yl)-2-oxo-2,3- dihydro-1H-imidazo[4,5-b]pyridin-1- yl)acetic acid | 485.0 |
| 36 | 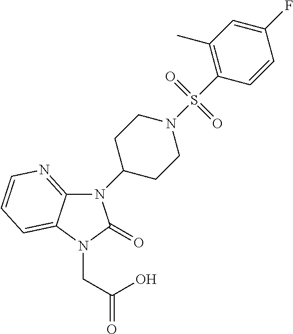 | 2-(3-(1-(4-fluoro-2-methylphenyl- sulfonyl)piperidin-4-yl)-2-oxo-2,3- dihydro-1H-imidazo[4,5-b]pyridin-1- yl)acetic acid | 449.0 |
| 37 | 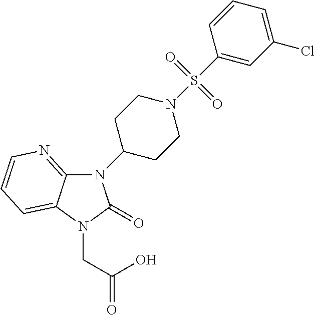 | 2-(3-(1-(3-chlorophenyl- sulfonyl)piperidin-4-yl)-2-oxo-2,3- dihydro-1H-imidazo[4,5-b]pyridin-1- yl)acetic acid | 451.0 |
| 38 | 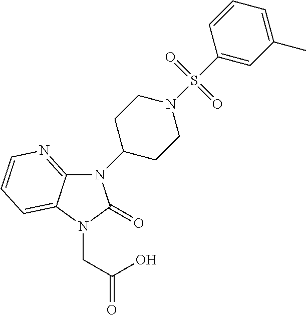 | 2-(2-oxo-3-(1-(m-tolylsulfonyl) piperidin-4-yl)-2,3-dihydro-1H- imidazo[4,5-b]pyridin-1-yl)acetic acid | 431.0 |
| 39 | 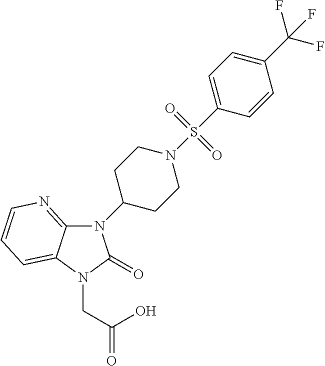 | 2-(2-oxo-3-(1-(4-(trifluoromethyl) phenylsulfonyl)piperidin-4-yl)-2,3- dihydro-1H-imidazo[4,5-b]pyridin-1- yl)acetic acid | 485.0 |
| 40 | 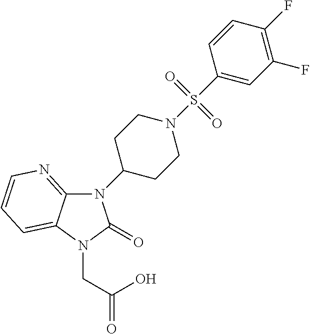 | 2-(3-(1-(3,4-difluorophenylsulfonyl) piperidin-4-yl)-2-oxo-2,3-dihydro-1H- imidazo[4,5-b]pyridin-1-yl)acetic acid | 453.0 |
| 41 | 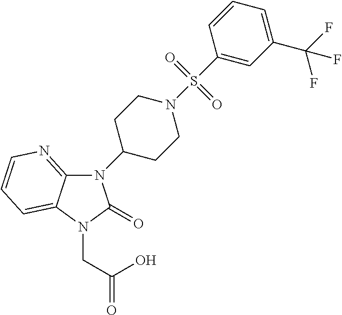 | 2-(2-oxo-3-(1-(3-(trifluoromethyl) phenylsulfonyl)piperidin-4-yl)-2,3- dihydro-1H-imidazo[4,5-b]pyridin-1- yl)acetic acid | 485.0 |
| 42 | 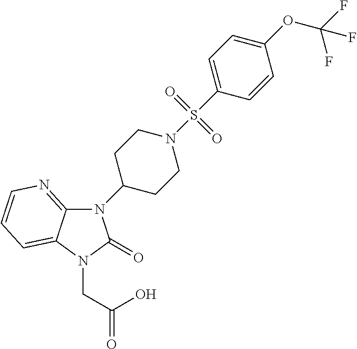 | 2-(2-oxo-3-(1-(4-(trifluoromethoxy) phenylsulfonyl)piperidin-4-yl)-2,3- dihydro-1H-imidazo[4,5-b]pyridin-1- yl)acetic acid | 501.0 |
| 43 | 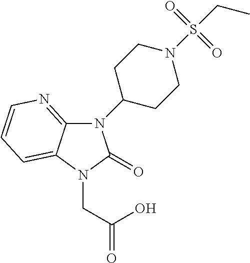 | 2-(3-(1-(ethylsulfonyl)piperidin-4-yl)- 2-oxo-2,3-dihydro-1H-imidazo[4,5- b]pyridin-1-yl)acetic acid | 369.1 |
| 44 | 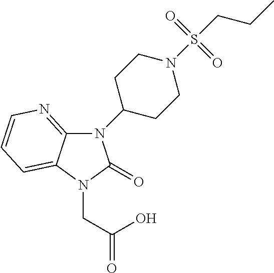 | 2-(2-oxo-3-(1-(propylsulfonyl) piperidin-4-yl)-2,3-dihydro-1H- imidazo[4,5-b]pyridin-1-yl)acetic acid | 383.0 |
| 45 | 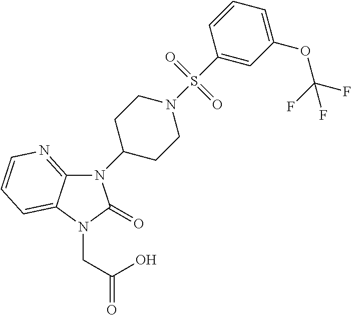 | 2-(2-oxo-3-(1-(3-(trifluoromethoxy) phenylsulfonyl)piperidin-4-yl)-2,3- dihydro-1H-imidazo[4,5-b]pyridin-1- yl)acetic acid | 501.0 |
| 46 | 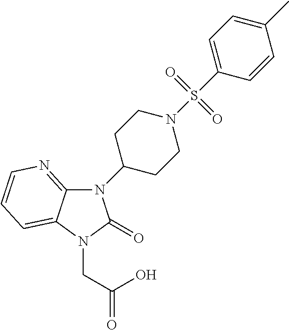 | 2-(2-oxo-3-(1-(p-tolylsulfonyl) piperidin-4-yl)-2,3-dihydro-1H- imidazo[4,5-b]pyridin-1-yl)acetic acid | 431.1 |
| 47 | 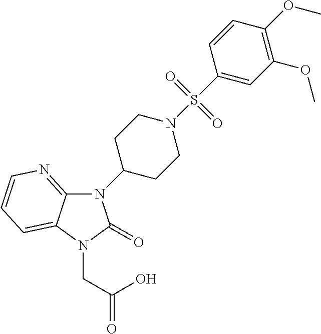 | 2-(3-(1-(3,4-dimethoxyphenyl- sulfonyl)piperidin-4-yl)-2-oxo-2,3- dihydro-1H-imidazo[4,5-b]pyridin-1- yl)acetic acid | 477.1 |
| 48 | 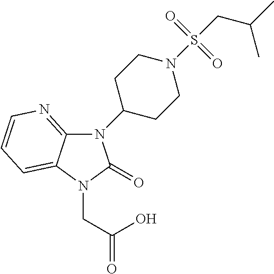 | 2-(3-(1-(isobutylsulfonyl)piperidin-4- yl)-2-oxo-2,3-dihydro-1H- imidazo[4,5-b]pyridin-1-yl)acetic acid | 397.1 |
| 49 | 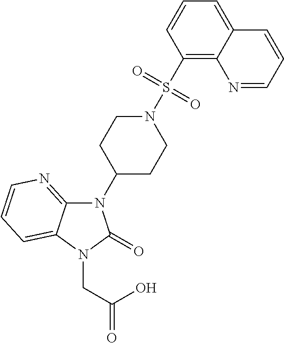 | 2-(2-oxo-3-(1-(quinolin-8- ylsulfonyl)piperidin-4-yl)-2,3- dihydro-1H-imidazo[4,5-b]pyridin-1- yl)acetic acid | 468.0 |
| 50 | 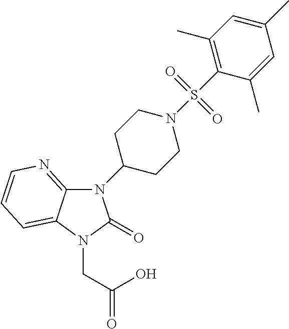 | 2-(3-(1-(mesitylsulfonyl)piperidin-4- yl)-2-oxo-2,3-dihydro-1H- imidazo[4,5-b]pyridin-1-yl)acetic acid | 459.1 |
| 51 | 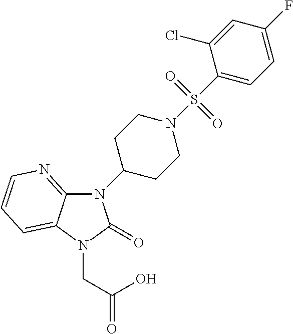 | 2-(3-(1-(2-chloro-4-fluorophenyl- sulfonyl)piperidin-4-yl)-2-oxo-2,3- dihydro-1H-imidazo[4,5-b]pyridin-1- yl)acetic acid | 469.0 |
| 52 | 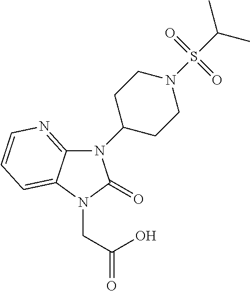 | 2-(3-(1-(isopropylsulfonyl)piperidin- 4-yl)-2-oxo-2,3-dihydro-1H- imidazo[4,5-b]pyridin-1-yl)acetic acid | 383.1 |
| 53 | 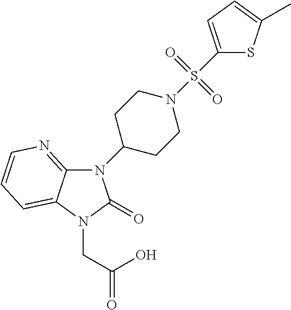 | 2-(3-(1-(5-methylthiophen-2- ylsulfonyl)piperidin-4-yl)-2-oxo-2,3- dihydro-1H-imidazo[4,5-b]pyridin-1- yl)acetic acid | 437.0 |
| 54 | 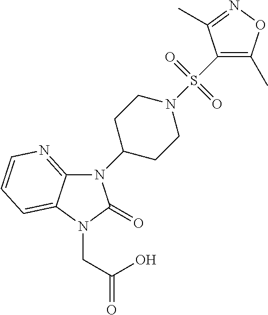 | 2-(3-(1-(3,5-dimethylisoxazol-4- ylsulfonyl)piperidin-4-yl)-2-oxo-2,3- dihydro-1H-imidazo[4,5-b]pyridin-1- yl)acetic acid | 436.0 |
| 55 | 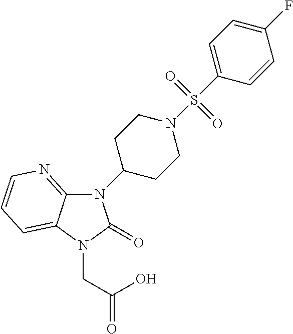 | 2-(3-(1-(4-fluorophenylsulfonyl) piperidin-4-yl)-2-oxo-2,3-dihydro-1H- imidazo[4,5-b]pyridin-1-yl)acetic acid | 435.0 |
| 56 | 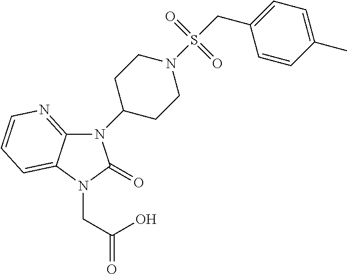 | 2-(3-(1-(4-methylbenzylsulfonyl) piperidin-4-yl)-2-oxo-2,3-dihydro-1H- imidazo[4,5-b]pyridin-1-yl)acetic acid | 445.0 |
| 57 | 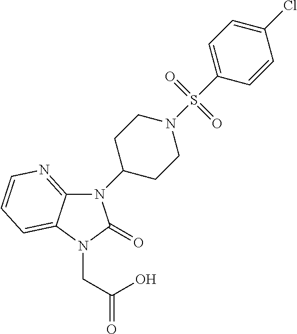 | 2-(3-(1-(4-chlorophenylsulfonyl) piperidin-4-yl)-2-oxo-2,3-dihydro-1H- imidazo[4,5-b]pyridin-1-yl)acetic acid | 451.0 |
| 58 | 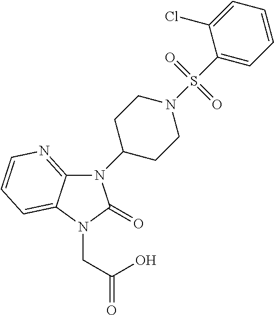 | 2-(3-(1-(2-chlorophenylsulfonyl) piperidin-4-yl)-2-oxo-2,3-dihydro-1H- imidazo[4,5-b]pyridin-1-yl)acetic acid | 451.0 |
| 59 | 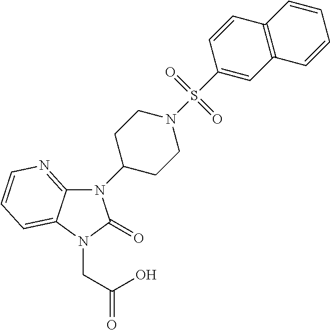 | 2-(3-(1-(naphthalen-2- ylsulfonyl)piperidin-4-yl)-2-oxo-2,3- dihydro-1H-imidazo[4,5-b]pyridin-1- yl)acetic acid | 467.0 |
| 60 | 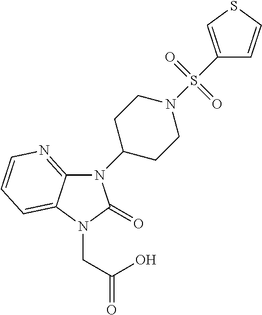 | 2-(2-oxo-3-(1-(thiophen-3-ylsulfonyl) piperidin-4-yl)-2,3-dihydro-1H- imidazo[4,5-b]pyridin-1-yl)acetic acid | 423.0 |
| 61 | 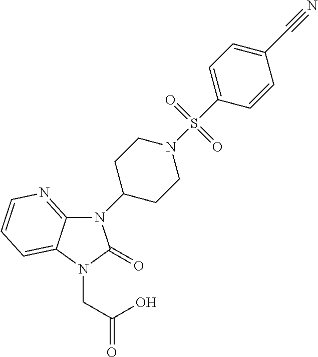 | 2-(3-(1-(4-cyanophenylsulfonyl) piperidin-4-yl)-2-oxo-2,3-dihydro-1H- imidazo[4,5-b]pyridin-1-yl)acetic acid | 442.0 |
| 62 | 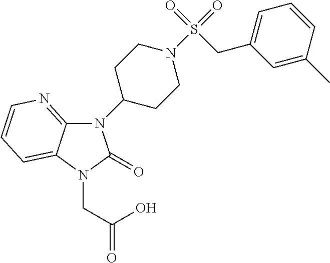 | 2-(3-(1-(3-methylbenzylsulfonyl) piperidin-4-yl)-2-oxo-2,3-dihydro-1H- imidazo[4,5-b]pyridin-1-yl)acetic acid | 445.1 |
| 63 | 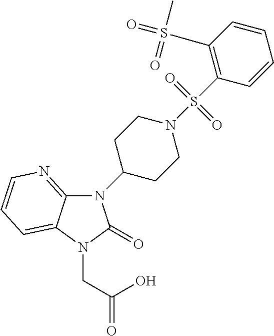 | 2-(3-(1-(2-(methylsulfonyl)phenyl- sulfonyl)piperidin-4-yl)-2-oxo-2,3- dihydro-1H-imidazo[4,5-b]pyridin-1- yl)acetic acid | 495.0 |
| 64 | 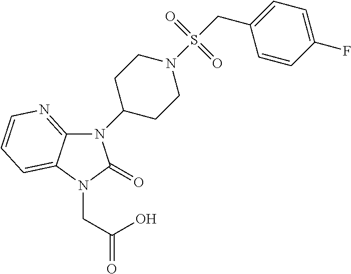 | 2-(3-(1-(4-fluorobenzylsulfonyl) piperidin-4-yl)-2-oxo-2,3-dihydro-1H- imidazo[4,5-b]pyridin-1-yl)acetic acid | 449.1 |
| 65 | 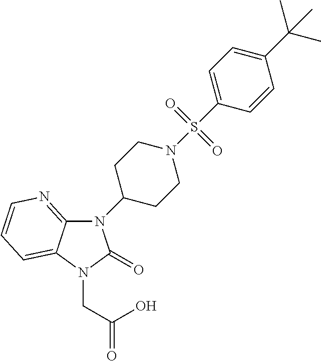 | 2-(3-(1-(4-tert-butylphenylsulfonyl) piperidin-4-yl)-2-oxo-2,3-dihydro-1H- imidazo[4,5-b]pyridin-1-yl)acetic acid | 473.1 |
| 66 | 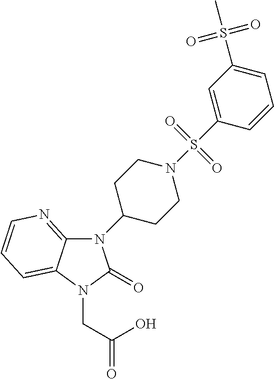 | 2-(3-(1-(3-(methylsulfonyl)phenyl- sulfonyl)piperidin-4-yl)-2-oxo-2,3- dihydro-1H-imidazo[4,5-b]pyridin-1- yl)acetic acid | 495.0 |
| 67 | 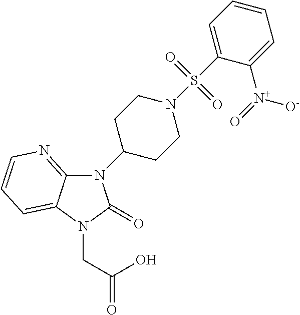 | 2-(3-(1-(2-nitrophenylsulfonyl) piperidin-4-yl)-2-oxo-2,3-dihydro-1H- imidazo[4,5-b]pyridin-1-yl)acetic acid | 462.0 |
| 68 | 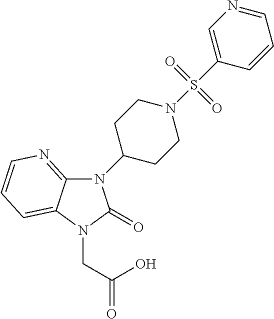 | 2-(2-oxo-3-(1-(pyridin-3-ylsulfonyl) piperidin-4-yl)-2,3-dihydro-1H- imidazo[4,5-b]pyridin-1-yl)acetic acid | 418.0 |
| 69 | 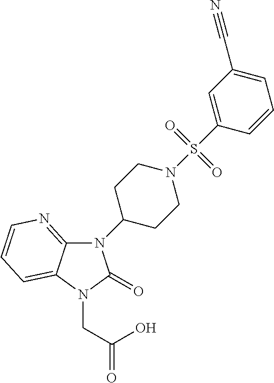 | 2-(3-(1-(3-cyanophenylsuIfonyl) piperidin-4-yl)-2-oxo-2,3-dihydro-1H- imidazo[4,5-b]pyridin-1-yl)acetic acid | 442.0 |
| 70 | 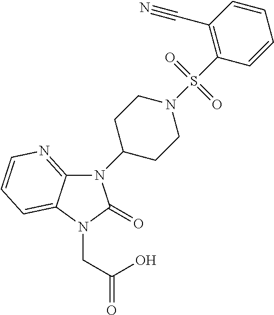 | 2-(3-(1-(2-cyanophenylsulfonyl) piperidin-4-yl)-2-oxo-2,3-dihydro-1H- imidazo[4,5-b]pyridin-1-yl)acetic acid | 442.0 |
| 71 | 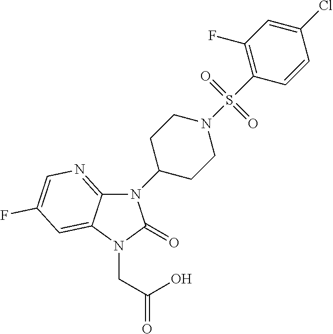 | 2-(3-(1-(4-chloro-2-fluorophenyl- sulfonyl)piperidin-4-yl)-6-fluoro-2- oxo-2,3-dihydro-1H-imidazo[4,5- b]pyridin-1-yl)acetic acid | 487.0 |
| 72 | 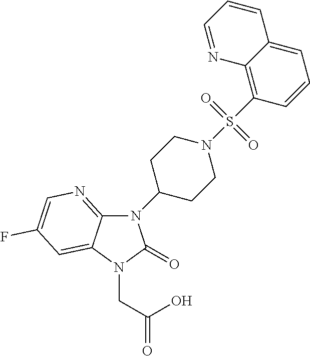 | 2-(6-fluoro-2-oxo-3-(1-(quinolin-8- ylsulfonyl)piperidin-4-yl)-2,3- dihydro-1H-imidazo[4,5-b]pyridin-1- yl)acetic acid | 486.0 |
| 73 | 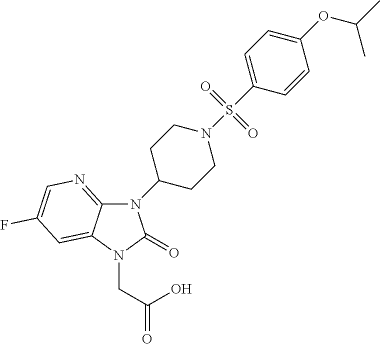 | 2-(6-fluoro-3-(1-(4-isopropoxyphenyl- sulfonyl)piperidin-4-yl)-2-oxo-2,3- dihydro-1H-imidazo[4,5-b]pyridin-1- yl)acetic acid | 493.0 |
| 74 | 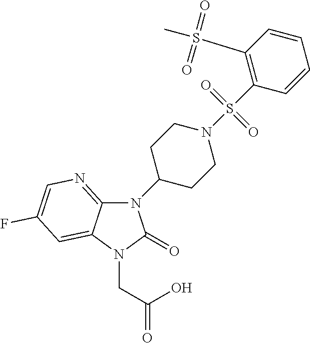 | 2-(6-fluoro-3-(1-(2-(methylsulfonyl) phenylsulfonyl)piperidin-4-yl)-2-oxo- 2,3-dihydro-1H-imidazo[4,5- b]pyridin-1-yl)acetic acid | 513.0 |
| 75 | 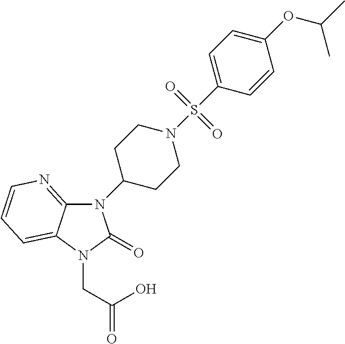 | 2-(3-(1-(4-isopropoxyphenylsulfonyl) piperidin-4-yl)-2-oxo-2,3-dihydro-1H- imidazo[4,5-b]pyridin-1-yl)acetic acid | 475.0 |
| 76 | 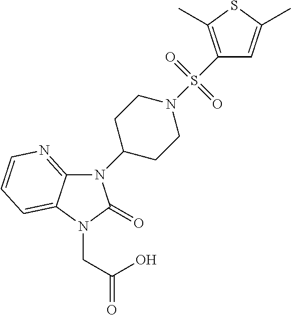 | 2-(3-(1-(2,5-dimethylthiophen-3- ylsulfonyl)piperidin-4-yl)-2-oxo-2,3- dihydro-1H-imidazo[4,5-b]pyridin-1- yl)acetic acid | 451.0 |
| 77 | 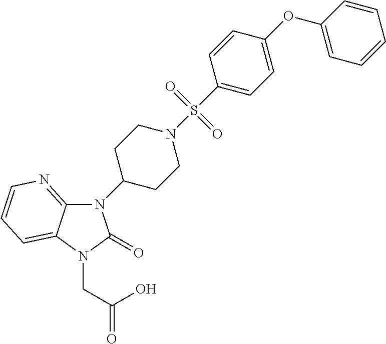 | 2-(2-oxo-3-(1-(4-phenoxyphenyl- sulfonyl)piperidin-4-yl)-2,3-dihydro- 1H-imidazo[4,5-b]pyridin-1-yl)acetic acid | 509.0 |
| 78 | 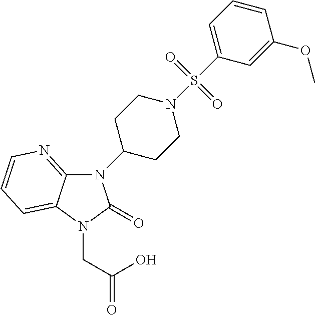 | 2-(3-(1-(3-methoxyphenyl- sulfonyl)piperidin-4-yl)-2-oxo-2,3- dihydro-1H-imidazo[4,5-b]pyridin-1- yl)acetic acid | 447.0 |
| 79 | 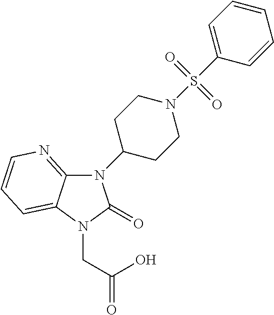 | 2-(2-oxo-3-(1-(phenylsulfonyl) piperidin-4-yl)-2,3-dihydro-1H- imidazo[4,5-b]pyridin-1-yl)acetic acid | 417.0 |
| 80 | 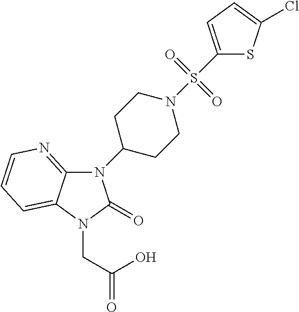 | 2-(3-(1-(5-chlorothiophen-2-yl- sulfonyl)piperidin-4-yl)-2-oxo-2,3- dihydro-1H-imidazo[4,5-b]pyridin-1- yl)acetic acid | 457.0 |
| 81 | 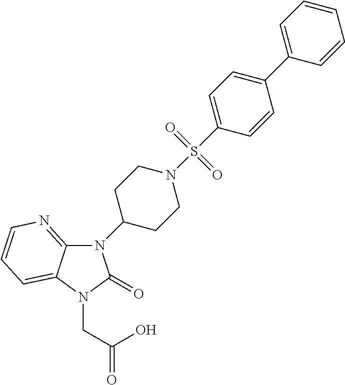 | 2-(3-(1-(biphenyl-4-ylsulfonyl) piperidin-4-yl)-2-oxo-2,3-dihydro-1H- imidazo[4,5-b]pyridin-1-yl)acetic acid | 493.0 |
| 82 | 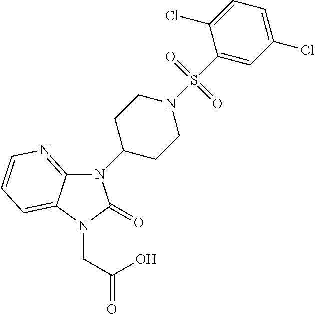 | 2-(3-(1-(2,5-dichlorophenylsulfonyl) piperidin-4-yl)-2-oxo-2,3-dihydro-1H- imidazo[4,5-b]pyridin-1-yl)acetic acid | 485.0 |
| 83 | 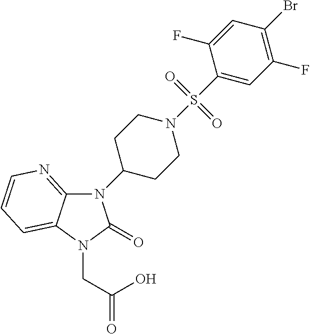 | 2-(3-(1-(4-bromo-2,5-difluorophenyl- sulfonyl)piperidin-4-yl)-2-oxo-2,3- dihydro-1H-imidazo[4,5-b]pyridin-1- yl)acetic acid | 533.0 |
| 84 | 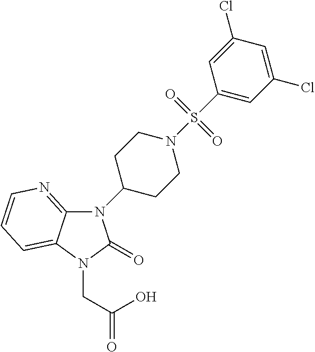 | 2-(3-(1-(3,5-dichlorophenyl- sulfonyl)piperidin-4-yl)-2-oxo-2,3- dihydro-1H-imidazo[4,5-b]pyridin-1- yl)acetic acid | 485.0 |
| 85 | 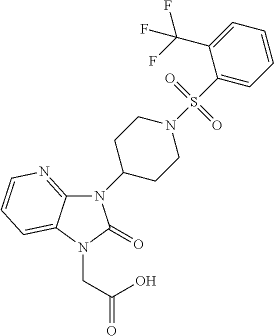 | 2-(2-oxo-3-(1-(2-(trifluoromethyl) phenylsulfonyl)piperidin-4-yl)-2,3- dihydro-1H-imidazo[4,5-b]pyridin-1- yl)acetic acid | 485.0 |
| 86 | 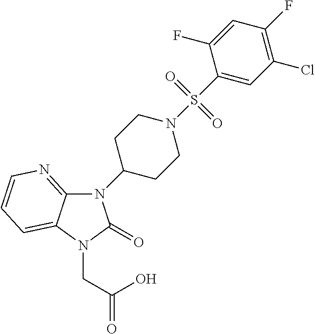 | 2-(3-(1-(5-chloro-2,4-difluorophenyl- sulfonyl)piperidin-4-yl)-2-oxo-2,3- dihydro-1H-imidazo[4,5-b]pyridin-1- yl)acetic acid | 487.0 |
| 87 | 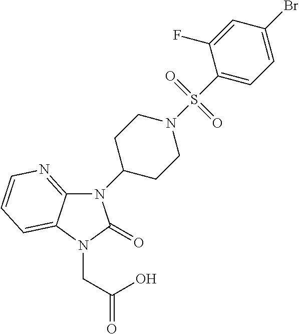 | 2-(3-(1-(4-bromo-2-fluorophenyl- sulfonyl)piperidin-4-yl)-2-oxo-2,3- dihydro-1H-imidazo[4,5-b]pyridin-1- yl)acetic acid | 515.0 |
| 88 | 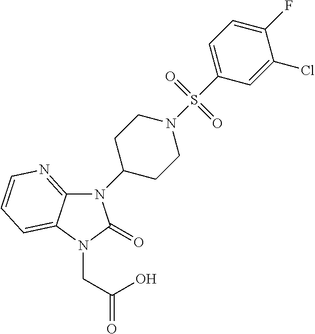 | 2-(3-(1-(3-chloro-4-fluorophenyl- sulfonyl)piperidin-4-yl)-2-oxo-2,3- dihydro-1H-imidazo[4,5-b]pyridin-1- yl)acetic acid | 469.0 |
| 89 | 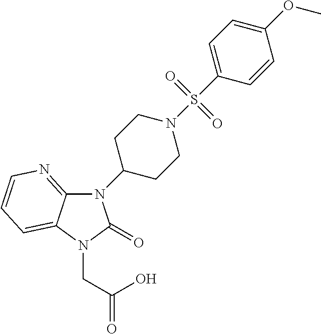 | 2-(3-(1-(4-methoxyphenylsulfonyl) piperidin-4-yl)-2-oxo-2,3-dihydro-1H- imidazo[4,5-b]pyridin-1-yl)acetic acid | 447.0 |
| 90 | 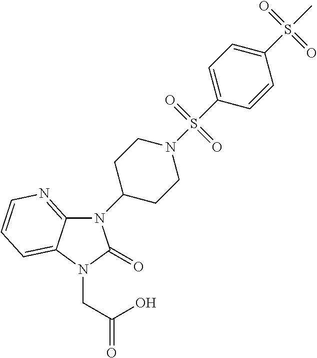 | 2-(3-(1-(4-(methylsulfonyl)phenyl- sulfonyl)piperidin-4-yl)-2-oxo-2,3- dihydro-1H-imidazo[4,5-b]pyridin-1- yl)acetic acid | 495.0 |
| 91 | 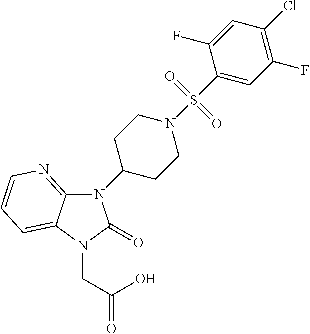 | 2-(3-(1-(4-chloro-2,5-difluoro- phenylsulfonyl)piperidin-4-yl)-2-oxo- 2,3-dihydro-1H-imidazo[4,5- b]pyridin-1-yl)acetic acid | 487.0 |
| 92 | 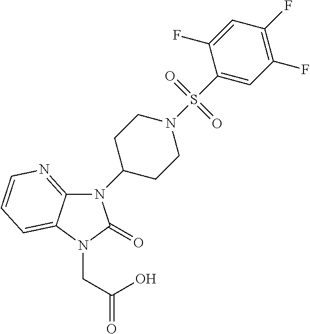 | 2-(2-oxo-3-(1-(2,4,5-trifluoro- phenylsulfonyl)piperidin-4-yl)-2,3- dihydro-1H-imidazo[4,5-b]pyridin-1- yl)acetic acid | 471.0 |
| 93 | 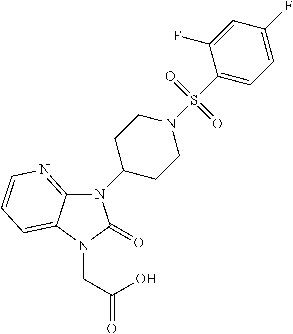 | 2-(3-(1-(2,4-difluorophenyl- sulfonyl)piperidin-4-yl)-2-oxo-2,3- dihydro-1H-imidazo[4,5-b]pyridin-1- yl)acetic acid | 453.0 |
| 94 | 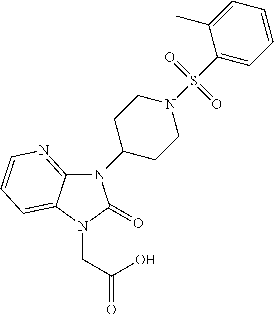 | 2-(2-oxo-3-(1-(o-tolylsulfonyl) piperidin-4-yl)-2,3-dihydro-1H- imidazo[4,5-b]pyridin-1-yl)acetic acid | 431.0 |
| 95 | 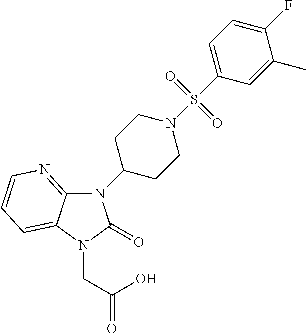 | 2-(3-(1-(4-fluoro-3- methylphenylsulfonyl)piperidin-4-yl)- 2-oxo-2,3-dihydro-1H-imidazo[4,5- b]pyridin-1-yl)acetic acid | 449 |
| 96 | 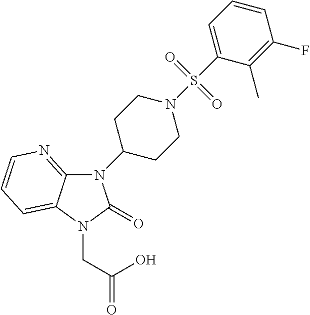 | 2-(3-(1-(3-fluoro-2- methylphenylsulfonyl)piperidin-4-yl)- 2-oxo-2,3-dihydro-1H-imidazo[4,5- b]pyridin-1-yl)acetic acid | 449 |
| 97 | 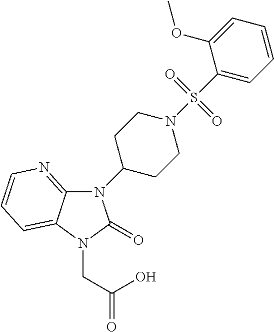 | 2-(3-(1-(2- methoxyphenylsulfonyl)piperidin-4- yl)-2-oxo-2,3-dihydro-1H- imidazo[4,5-b]pyridin-1-yl)acetic acid | 447 |
| 98 | 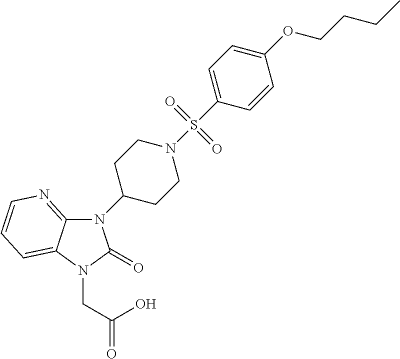 | 2-(3-(1-(4- butoxyphenylsulfonyl)piperidin-4-yl)- 2-oxo-2,3-dihydro-1H-imidazo[4,5- b]pyridin-1-yl)acetic acid | 489 |
| 99 | 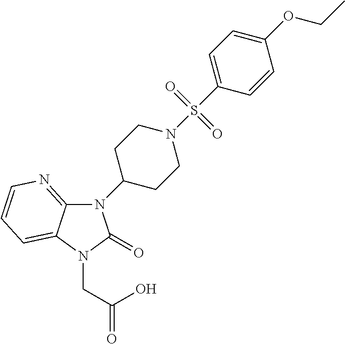 | 2-(3-(1-(4- ethoxyphenylsulfonyl)piperidin-4-yl)- 2-oxo-2,3-dihydro-1H-imidazo[4,5- b]pyridin-1-yl)acetic acid | 461 |
| 100 | 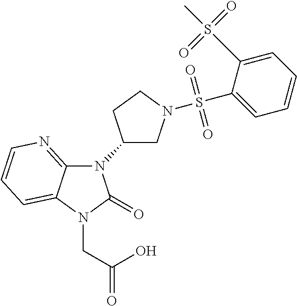 | (R)-2-(3-(1-(2- (methylsulfonyl)phenylsulfonyl) pyrrolidin-3-yl)-2-oxo-2,3-dihydro-1H- imidazo[4,5-b]pyridin-1-yl)acetic acid | 481 |
| 101 | 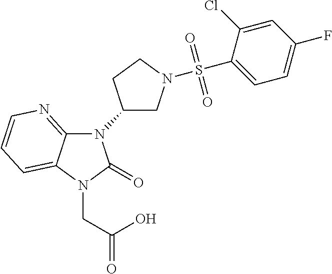 | (R)-2-(3-(1-(2-chloro-4- fluorophenylsulfonyl)pyrrolidin-3-yl)- 2-oxo-2,3-dihydro-1H-imidazo[4,5- b]pyridin-1-yl)acetic acid | 455 |
| 102 | 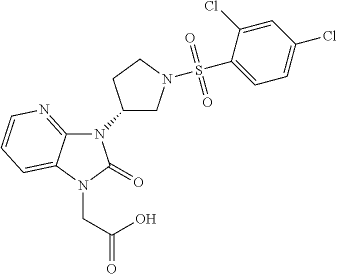 | (R)-2-(3-(1-(2,4- dichlorophenylsulfonyl)pyrrolidin-3- yl)-2-oxo-2,3-dihydro-1H- imidazo[4,5-b]pyridin-1-yl)acetic acid | 471 |
| 103 | 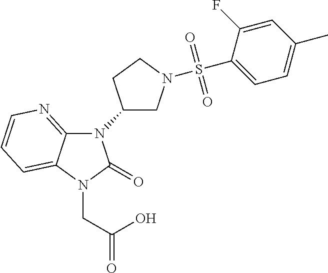 | (R)-2-(3-(1-(2-fluoro-4- methylphenylsulfonyl)pyrrolidin-3- yl)-2-oxo-2,3-dihydro-1H- imidazo[4,5-b]pyridin-1-yl)acetic acid | 435 |
| 104 | 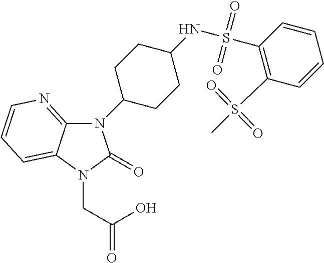 | 2-(3-(4-(2- (methylsulfonyl)phenylsulfonamido) cyclohexyl)-2-oxo-2,3-dihydro-1H- imidazo[4,5-b]pyridin-1-yl)acetic acid | 509 |
| 105 | 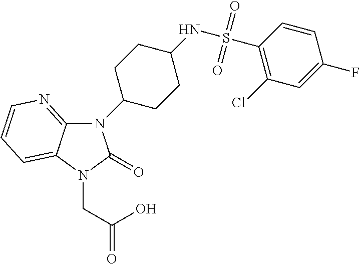 | 2-(3-(4-(2-chloro-4- fluorophenylsulfonamido)cyclohexyl)- 2-oxo-2,3-dihydro-1H-imidazo[4,5- b]pyridin-1-yl)acetic acid | 483 |
| 106 | 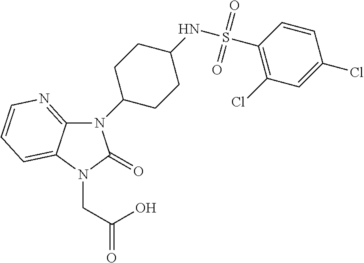 | 2-(3-(4-(2,4- dichlorophenylsulfonamido) cyclohexyl)-2-oxo-2,3-dihydro-1H- imidazo[4,5-b]pyridin-1-yl)acetic acid | 499 |
| 107 | 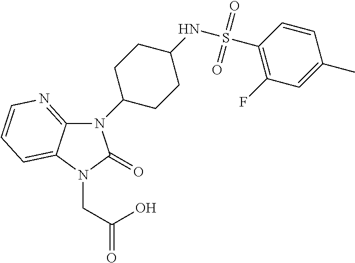 | 2-(3-(4-(2-fluoro-4- methylphenylsulfonamido)cyclohexyl)- 2-oxo-2,3-dihydro-1H-imidazo[4,5- b]pyridin-1-yl)acetic acid | 463 |
| 108 | 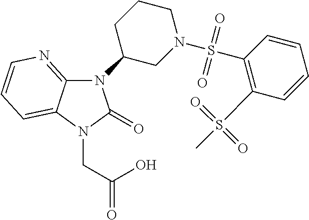 | (S)-2-(3-(1-(2- (methylsulfonyl)phenylsulfonyl) piperidin-3-yl)-2-oxo-2,3-dihydro-1H- imidazo[4,5-b]pyridin-1-yl)acetic acid | 495 |
| 109 | 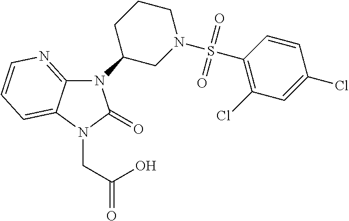 | (S)-2-(3-(1-(2,4- dichlorophenylsulfonyl)piperidin-3- yl)-2-oxo-2,3-dihydro-1H- imidazo[4,5-b]pyridin-1-yl)acetic acid | 485 |
| 110 | 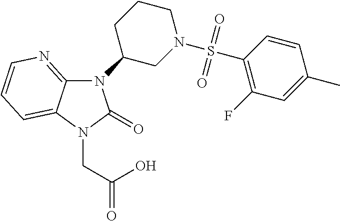 | (S)-2-(3-(1-(2-fluoro-4- methylphenylsulfonyl)piperidin-3-yl)- 2-oxo-2,3-dihydro-1H-imidazo[4,5- b]pyridin-1-yl)acetic acid | 449 |
| 111 | 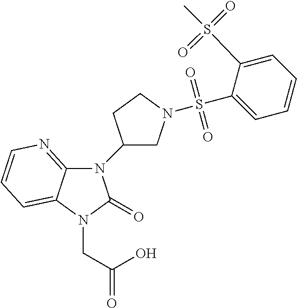 | 2-(3-(1-(2- (methylsulfonyl)phenylsulfonyl) pyrrolidin-3-yl)-2-oxo-2,3-dihydro-1H- imidazo[4,5-b]pyridin-1-yl)acetic acid | 481 |
| 112 | 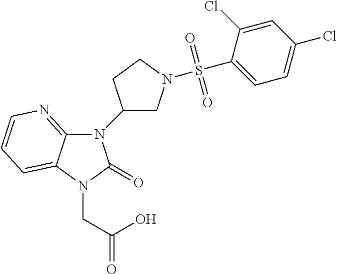 | 2-(3-(1-(2,4- dichlorophenylsulfonyl)pyrrolidin-3- yl)-2-oxo-2,3-dihydro-1H- imidazo[4,5-b]pyridin-1-yl)acetic acid | 471 |
| 113 | 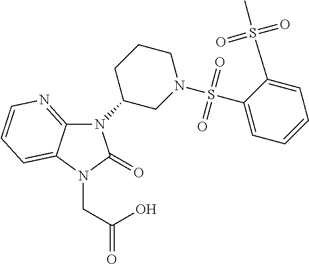 | (R)-2-(3-(1-(2- (methylsulfonyl)phenylsulfonyl) piperidin-3-yl)-2-oxo-2,3-dihydro-1H- imidazo[4,5-b]pyridin-1-yl)acetic acid | 495 |
| 114 | 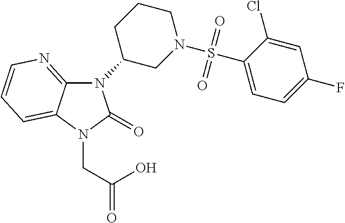 | (R)-2-(3-(1-(2-chloro-4- fluorophenylsulfonyl)piperidin-3-yl)- 2-oxo-2,3-dihydro-1H-imidazo[4,5- b]pyridin-1-yl)acetic acid | 469 |
| 115 | 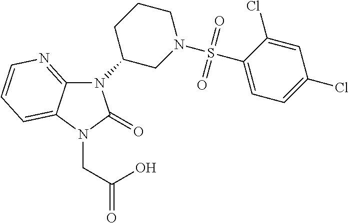 | (R)-2-(3-(1-(2,4- dichlorophenylsulfonyl)piperidin-3- yl)-2-oxo-2,3-dihydro-1H- imidazo[4,5-b]pyridin-1-yl)acetic acid | 485 |
| 116 | 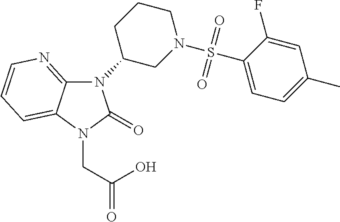 | (R)-2-(3-(1-(2-fluoro-4- methylphenylsulfonyl)piperidin-3-yl)- 2-oxo-2,3-dihydro-1H-imidazo[4,5- b]pyridin-1-yl)acetic acid | 449 |
| 117 | 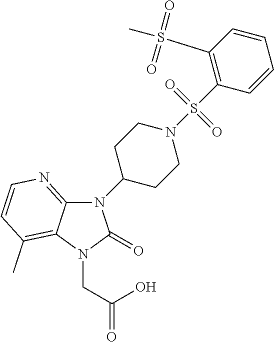 | 2-(7-methyl-3-(1-(2- (methylsulfonyl)phenylsulfonyl) piperidin-4-yl)-2-oxo-2,3-dihydro-1H- imidazo[4,5-b]pyridin-1-yl)acetic acid | 509 |
| 118 | 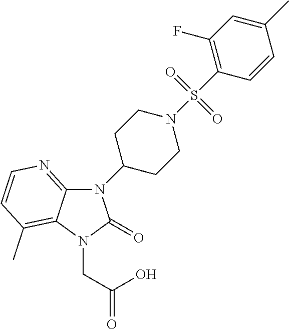 | 2-(3-(1-(2-fluoro-4- methylphenylsulfonyl)piperidin-4-yl)- 7-methyl-2-oxo-2,3-dihydro-1H- imidazo[4,5-b]pyridin-1-yl)acetic acid | 463 |
| 119 | 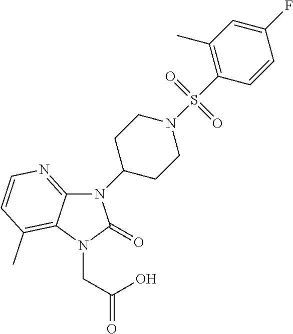 | 2-(3-(1-(4-fluoro-2- methylphenylsulfonyl)piperidin-4-yl)- 7-methyl-2-oxo-2,3-dihydro-1H- imidazo[4,5-b]pyridin-1-yl)acetic acid | 463 |
| 120 | 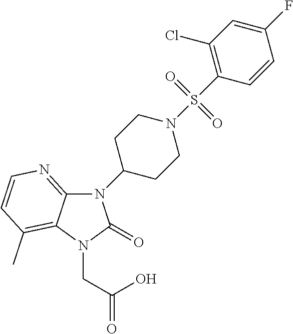 | 2-(3-(1-(2-chloro-4- fluorophenylsulfonyl)piperidin-4-yl)- 7-methyl-2-oxo-2,3-dihydro-1H- imidazo[4,5-b]pyridin-1-yl)acetic acid | 483 |
| 121 | 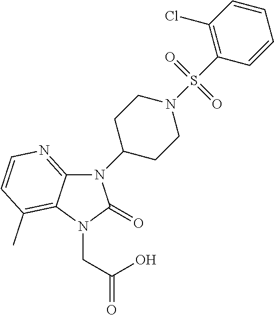 | 2-(3-(1-(2- chlorophenylsulfonyl)piperidin-4-yl)- 7-methyl-2-oxo-2,3-dihydro-1H- imidazo[4,5-b]pyridin-1-yl)acetic acid | 465 |
| 122 | 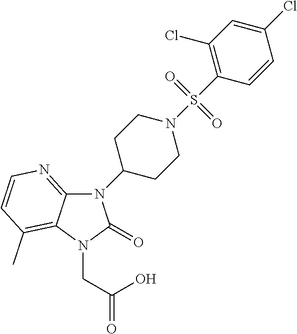 | 2-(3-(1-(2,4- dichlorophenylsulfonyl)piperidin-4- yl)-7-methyl-2-oxo-2,3-dihydro-1H- imidazo[4,5-b]pyridin-1-yl)acetic acid | 499 |
| 123 | 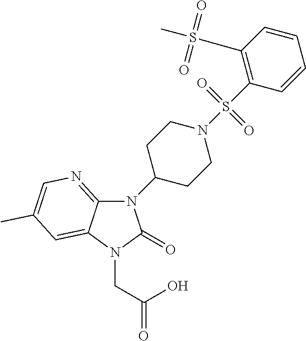 | 2-(6-methyl-3-(1-(2- (methylsulfonyl)phenylsulfonyl) piperidin-4-yl)-2-oxo-2,3-dihydro-1H- imidazo[4,5-b]pyridin-1-yl)acetic acid | 509 |
| 124 | 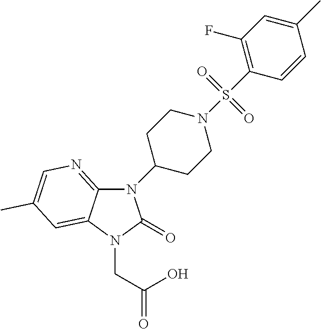 | 2-(3-(1-(2-fluoro-4- methylphenylsulfonyl)piperidin-4-yl)- 6-methyl-2-oxo-2,3-dihydro-1H- imidazo[4,5-b]pyridin-1-yl)acetic acid | 463 |
| 125 | 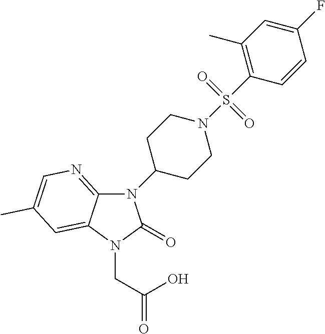 | 2-(3-(1-(4-fluoro-2- methylphenylsulfonyl)piperidin-4-yl)- 6-methyl-2-oxo-2,3-dihydro-1H- imidazo[4,5-b]pyridin-1-yl)acetic acid | 463 |
| 126 | 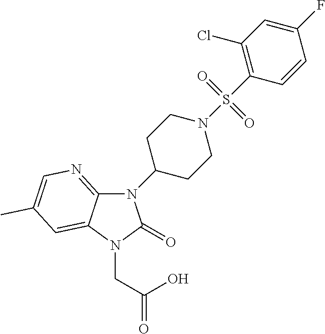 | 2-(3-(1-(2-chloro-4- fluorophenylsulfonyl)piperidin-4-yl)- 6-methyl-2-oxo-2,3-dihydro-1H- imidazo[4,5-b]pyridin-1-yl)acetic acid | 483 |
| 127 | 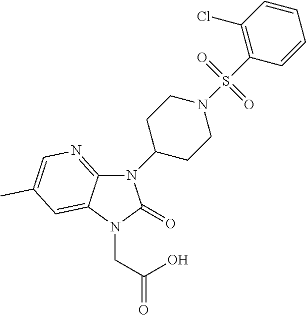 | 2-(3-(1-(2- chlorophenylsulfonyl)piperidin-4-yl)- 6-methyl-2-oxo-2,3-dihydro-1H- imidazo[4,5-b]pyridin-1-yl)acetic acid | 465 |

Synthetic Scheme 7

The method used for synthesis of compounds 132-135 is depicted in Scheme 7.

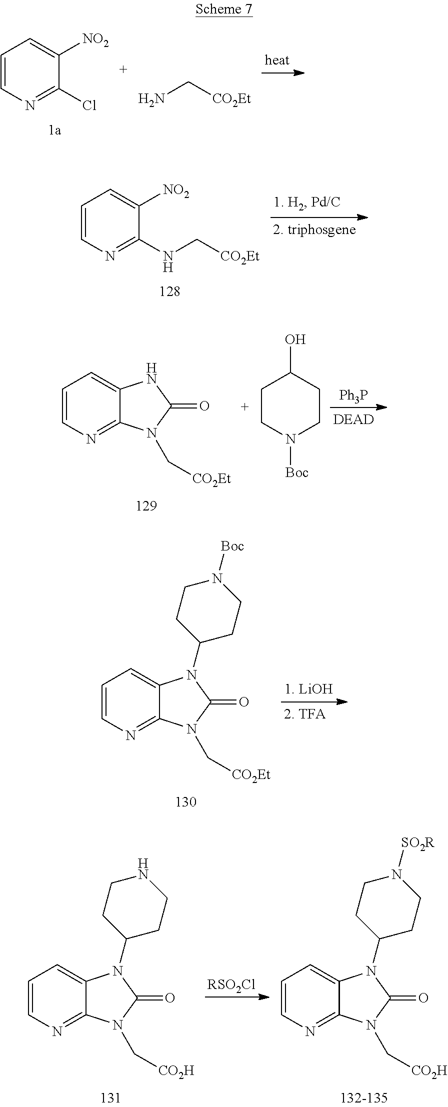

2-Chloro-3-nitropyridine 1a was heated with glycine ethyl ester under microwave irradiation to give intermediate 128. Reduction of the nitro group followed by ring closure with triphosgene provided bicyclic intermediate 129. A Mitsunobu reaction between intermediate 129 and tert-butyl 4-hydroxypiperidine-1-carboxylate yielded the ethyl ester intermediate, 130. The ethyl ester was hydrolyzed by lithium hydroxide and the Boc group was removed with TFA. Reaction of intermediate 131 with sulfonyl chlorides provided compounds 132-135.

### Intermediate 131: Preparation of 2-(2-oxo-1-(piperidin-4-yl)-1H-imidazo[4,5-b]pyridin-3(2H)-yl)acetic acid (131)

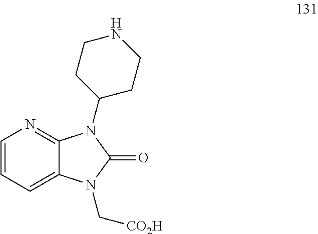

### Step 1: Preparation of ethyl 2-(3-nitropyridin-2-ylamino)acetate (128).

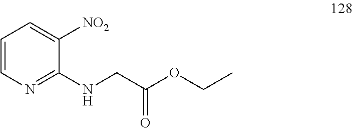

A mixture of 2-chloro-3-nitropyridine (1.75 g), ethyl-2-amino-acetate (1.71 g) and DIEA (4.0 mL) in acetonitrile was heated by microwave irradiation at 150° C. for 30 min. After evaporation of acetonitrile, the residue was extracted between EtOAc and aqueous sodium bicarbonate. The organic phase was dried over sodium sulfate and evaporated to dryness. The residue was purified by column chromatography with a gradient of 15% to 40% EtOAc/hexanes to give ethyl 2-(3-nitropyridin-2-ylamino)acetate as a solid (yield: 2.35 g). LC-MS (+ESI) m/z=226 [M+H]⁺.

### Step 2: Preparation of ethyl 2-(3-aminopyridin-2-ylamino)acetate

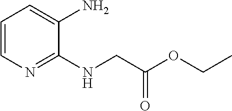

A mixture of 2-(3-nitropyridin-2-ylamino)acetate (2.35 g), palladium on carbon (10%, 0.42 g) in methanol was hydrogenated under hydrogen (60 psi) in a Parr shaker for 2 hours. The catalyst was removed by filtration through celite. Evaporation of the filtrate under vacuum provided ethyl 2-(3-aminopyridin-2-ylamino)acetate as a solid (yield: 2.04 g), which was used in Step 3 without further purification. LC-MS (+ESI) m/z=196 [M+H]⁺.

### Step 3: Preparation of ethyl 2-(2-oxo-1H-imidazo[4,5-b]pyridin-3(2H)-yl)acetate (129)

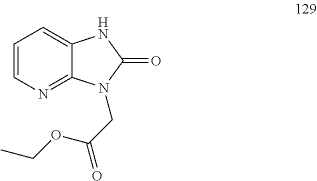

Trisphogene (1.1 g) was added portionwise to a solution of ethyl 2-(3-aminopyridin-2-ylamino)acetate as a solid (2.04 g) and DIEA (3.5 mL) in DCM (40 mL) at 0° C. After stirring at 0° C. for 30 min, aqueous sodium bicarbonate was added and stirred at room temperature for 15 min. The organic phase was separated and dried over sodium sulfate. After evaporation of DCM, the residue was purified by column chromatography with 70% to 100% EtOAc/Hex to give ethyl 2-(2-oxo-1H-imidazo[4,5-b]pyridin-3(2H)-yl)acetate as a solid (Yield: 1.66 g). LC-MS (+ESI) m/z=222 [M+H]⁺.

### Step 4: Preparation of tert-butyl 4-(3-(2-ethoxy-2-oxoethyl)-2-oxo-2,3-dihydro-1H-imidazo[4,5-b]pyridin-1-yl)piperidine-1-carboxylate (130)

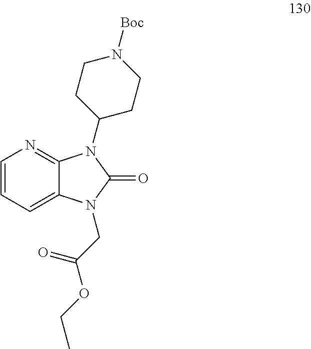

To a solution of tert-butyl 4-hydroxypiperidine-1-carboxylate (1.34 g), triphenyl-phosphine (1.75 g) and diethylazodicarboxylate (2.64 mL) in anhydrous toluene (15 mL) was added ethyl 2-oxo-1H-imidazo[4,5-b]pyridine-3(2H)-carboxylate (1.15 g). After stirring for 4 hours, the organic solvent was evaporated to dryness. The residue was taken up in DCM and washed with sodium bicarbonate. The organic phase was separated, dried over sodium sulfate and evaporated to dryness. The residue was purified by column chromatography with 20% to 80% EtOAc/hexanes and then by reverse phase column chromatography with 10% to 100% CH₃CN/H₂O to provide tert-butyl 44342-ethoxy-2-oxoethyl)-2-oxo-2,3-dihydro-1H-imidazo[4,5-b]pyridin-1-yl)piperidine-1-carboxylate as a solid (yield: 1.3 g). LC-MS (+ESI) m/z=405 [M+H]⁺.

### Step 5: Preparation of 2-(1-(1-(tert-butoxycarbonyl)piperidin-4-yl)-2-oxo-1H-imidazo[4,5-b]pyridin-3(2H)-yl)acetic acid

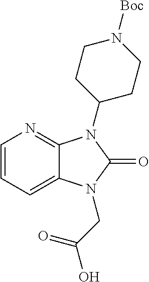

A solution of 2-(1-(1-(tert-butoxycarbonyl)piperidin-4-yl)-2-oxo-1H-imidazo[4,5-b]pyridin-3(2H)-yl)acetic acid (0.3 g) was stirred in LiOH/H₂O (2M, 1 mL) at room temperature for 2 hours. After evaporation of MeOH, the aqueous layer was acidified to pH 2 with concentrated HCl and extracted with 10% isopropanol/DCM. Evaporation of the solvent provided 2-(1-(1-(tert-butoxycarbonyl)piperidin-4-yl)-2-oxo-1H-imidazo[4,5-b]pyridin-3(2H)-yl)acetic acid as a solid (0.26 g). LC-MS (+ESI) m/z=377 [M+H]⁺.

### Step 6: Preparation of 2-(2-oxo-1-(piperidin-4-yl)-1H-imidazo[4,5-b]pyridin-3(2H)-yl)acetic acid (131)

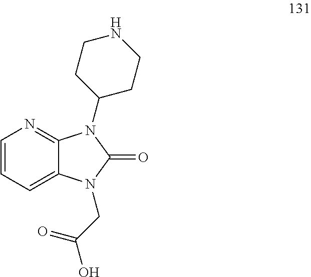

A solution of 2-(1-(1-(tert-butoxycarbonyl)piperidin-4-yl)-2-oxo-1H-imidazo[4,5-b]pyridin-3(2H)-yl)acetic acid (0.26 g) was stirred in TFA/DCM (3:1) at room temperature for 2 hours. After evaporation of TFA and DCM, the residue was azeotroped with toluene twice to provide the TFA salt of 2-(2-oxo-1-(piperidin-4-yl)-1H-imidazo[4,5-b]pyridin-3(2H)-yl)acetic acid as an oil. LC-MS (+ESI) m/z=277 [M+H]⁺.

### Example 2

### Preparation of 2-(1-(1-(2-chloro-4-fluorophenylsulfonyl)piperidin-4-yl)-2-oxo-1H-imidazo[4,5-b]pyridin-3(2H)-yl)acetic acid (132)

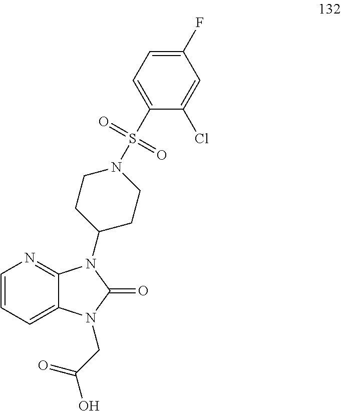

To a solution of 2-(2-oxo-1-(piperidin-4-yl)-1H-imidazo[4,5-b]pyridin-3(2H)-yl)acetic acid (40 mg) in THF/water (1:1) was added 2-chloro-4-fluorobenzenesulfonyl chloride (40 μL). After stirring at room temperature for 2 hours, the reaction mixture was concentrated and diluted with methanol (0.5 mL). The reaction mixture was purified by prep LC-MS with a gradient from 5% MeCN/water to 95% MeCN/water run over 14 minutes. Pure fractions were evaporated to provide 2-(1-(1-(2-chloro-4-fluorophenyl-sulfonyl)piperidin-4-yl)-2-oxo-1H-imidazo[4,5-b]pyridin-3(2H)-yl)acetic acid (yield: 4.7 mg). LC-MS (+ESI) m/z=469 [M+H]⁺.

Compounds 133-135 were prepared according to the procedure described for the synthesis of compound 132 by reaction between intermediate 131 with 2-chlorobenzene-sulfonyl chloride, 2,4-dichlorobenzenesulfonyl chloride and 2-methylsulfonylbenzene-sulfonyl chloride, respectively.
2-(1-(1-(2-chlorophenylsulfonyl)piperidin-4-yl)-2-oxo-1H-imidazo[4,5-b]pyridin-3(2H)-yl)acetic acid (133). LC-MS (+ESI) m/z=451 [M+H]⁺.
2-(1-(1-(2,4-dichlorophenylsulfonyl)piperidin-4-yl)-2-oxo-1H-imidazo[4,5-b]pyridin-3(2H)-yl)acetic acid (134). LC-MS (+ESI) m/z=485 [M+H]⁺.
2-(1-(1-(2-(methylsulfonyl)phenyl-sulfonyl)piperidin-4-yl)-2-oxo-1H-imidazo[4,5-b]pyridin-3(2H)-yl)acetic acid (135). LC-MS (+ESI) m/z=495 [M+H]⁺.

The synthesis of compound 140 is depicted in Scheme 8 (See below).

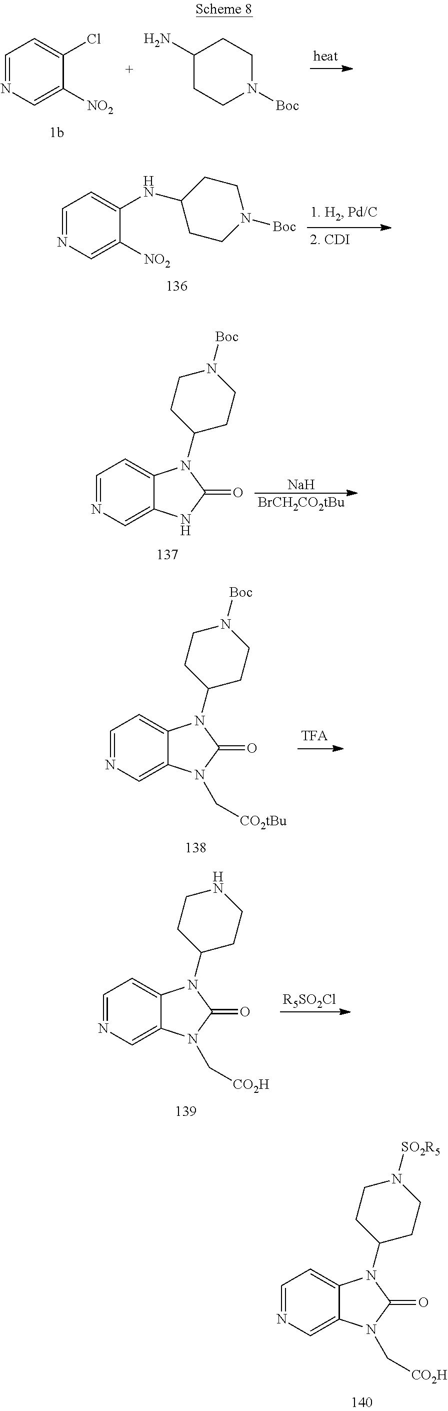

4-Chloro-3-nitropyridine 1b was heated with tert-butyl 4-aminopiperidine-1-carboxylate under microwave irradiation to give intermediate 136. Reduction of nitro group followed by ring closure with CDI yielded intermediate 137. Treatment of intermediate 137 with sodium hydride followed by addition of tert-butyl bromoacetate gave intermediate 138. Deprotection of Boc group and tert-butyl ester was achieved by treatment with TFA in DCM to provide intermediate 139, which was reacted with 2-chloro-4-fluorobenzenesulfonyl chloride to give compound 140.

### Intermediate 156: Preparation of 2-(2-oxo-1-(piperidin-4-yl)-1H-imidazo[4,5-c]pyridin-3(2H)-yl)acetic acid (139)

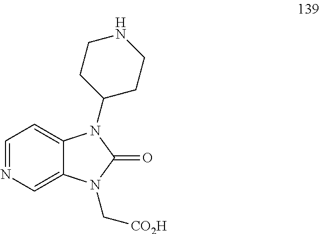

### Step 1: Preparation of tert-butyl 4-(3-nitropyridin-4-ylamino)piperidine-1-carboxylate (136)

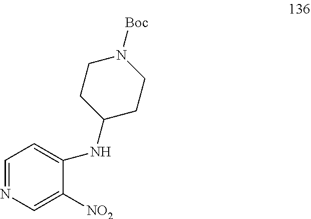

A mixture of 4-chloro-3-nitropyridine (1.0 g), tert-butyl 4-aminopiperidine-1-carboxylate (1.5 g) and DIEA (2.2 mL) in acetonitrile was heated by microwave irradiation at 135° C. for 30 min. After evaporation of acetonitrile, the residue was extracted between EtOAc and aqueous sodium bicarbonate. The organic phase was dried over sodium sulfate and evaporated to give tert-butyl 4-(3-nitropyridin-4-ylamino) piperidine-1-carboxylate as a solid (yield: 2.01 g), which is used in Step 2 without further purification. LC-MS (+ESI) m/z=323 [M+H]⁺.

### Step 2: Preparation of tert-butyl 4-(3-aminopyridin-4-ylamino)piperidine-1-carboxylate

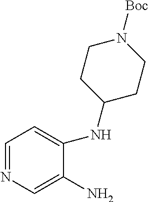

A mixture of tert-butyl 4-(3-nitropyridin-4-ylamino)piperidine-1-carboxylate (2.01 g), palladium on carbon (10%, 0.67 g) in methanol was hydrogenated under hydrogen (60 psi) in a Parr shaker for 1 hour. The catalyst was removed by filtration through celite. Evaporation of the filtrate under vacuum give tert-butyl 4-(3-amino-pyridin-4-ylamino)piperidine-1-carboxylate (yield: 1.8 g), which is used in Step 3 without further purification. LC-MS (+ESI) m/z=293 [M+H]⁺.

### Step 3: Preparation of tert-butyl 4-(2-oxo-2,3-dihydro-1H-imidazo[4,5-c]pyridin-1-yl)piperidine-1-carboxylate (137)

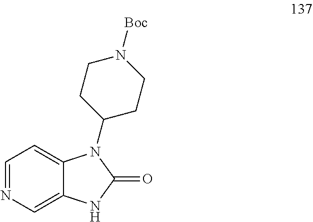

CDI (1.6 g) was added in portions to a solution of tert-butyl-4-(3-aminopyridin-4-ylamino)piperidine-1-carboxylate (1.8 g) and DIEA (2.3 mL) in DCM (20 mL). After stirring at room temperature for 3 hours, the reaction was washed with 1N HCl (10 mL). The organic phase was separated and dried over sodium sulfate. After evaporation of DCM, the residue was purified by column chromatography with 40% to 80% EtOAc/hex to give tert-butyl 4-(2-oxo-2,3-dihydro-1H-imidazo[4,5-c]pyridin-1-yl)piperidine-1-carboxylate as a solid (Yield: 1.52 g). LC-MS (+ESI) m/z=319 [M+H]⁺.

### Step 4: Preparation of tert-butyl 4-(3-(2-tert-butoxy-2-oxoethyl)-2-oxo-2,3-dihydro-1H-imidazo[4,5-c]pyridin-1-yl)piperidine-1-carboxylate (138)

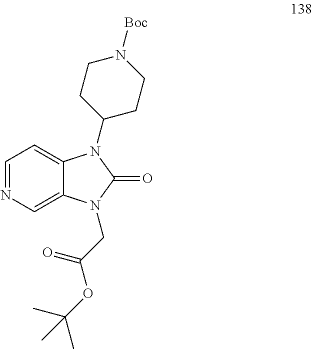

To a solution of tert-butyl 4-(2-oxo-2,3-dihydro-1H-imidazo[4,5-c]pyridin-1-yl)piperidine-1-carboxylate (1.52 g) in anhydrous THF (30 mL) was added sodium hydride (0.22 g, 60% dispersion) at 0° C. After stirring at 0° C. for 10 min, tert-butyl bromoacetate (1.8 g) was added and the mixture was stirred at 0° C. for 1 hour. The reaction was quenched with aqueous ammonium chloride and extracted with EtOAc. The organic phase was dried over sodium sulfate and evaporated to dryness. The residue was purified by column chromatography with 20% to 50% EtOAc/hex to give tert-butyl 4-(3-(2-tert-butoxy-2-oxoethyl)-2-oxo-2,3-dihydro-1H-imidazo[4,5-c]pyridin-1-yl)piperidine-1-carboxylate as a solid (yield: 1.12 g). ¹H NMR (CDCl₃) δ 8.03 (dd, J=1.3, 5.2 Hz, 1H), 7.06 (dd, J=1.4, 7.7 Hz, 1H), 6.98 (dd, J=5.2, Hz, 1H), 4.54-4.58 (m, 1H), 4.52 (s, 2H), 4.29 (br, 2H), 2.84 (br, 2H), 2.62-2.69 (m, 2H), 1.76-1.79 (m, 2H), 1.48 (s, 9H), 1.46 (s, 9H). LC-MS (+ESI) m/z=433 [M+H]⁺.

### Step 5: Preparation of 2-(2-oxo-1-(piperidin-4-yl)-1H-imidazo[4,5-c]pyridin-3(2H)-yl)acetic acid. (139).

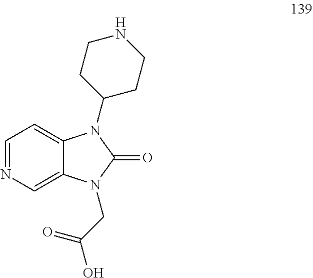

A solution of tert-butyl 4-(3-(2-tert-butoxy-2-oxoethyl)-2-oxo-2,3-dihydro-1H-imidazo[4,5-c]pyridin-1-yl)piperidine-1-carboxylate (1.12 g) was stirred in TFA/DCM (3:1) at room temperature for 2 hours. After evaporation of TFA and DCM, the residue was azeotroped with toluene twice to give the TFA salt of 2-(2-oxo-1-(piperidin-4-yl)-1H-imidazo[4,5-c]pyridin-3(2H)-yl)acetic acid (0.68 g) as an oil. LC-MS (+ESI) m/z=277 [M+H]⁺.

### Example 3

### Preparation of 2-(1-(1-(2-chloro-4-fluorophenylsulfonyl)piperidin-4-yl)-2-oxo-1H-imidazo[4,5-c]pyridin-3(2H)-yl)acetic acid (140)

To a solution of 2-(2-oxo-1-(piperidin-4-yl)-1H-imidazo[4,5-c]pyridin-3(2H)-yl)acetic acid 139 (28 mg) in THF/water (1:1) was added 2-chloro-4-fluorobenzene-sulfonyl chloride (30 μL). After stirring at room temperature for 2 hours, the reaction mixture was concentrated and diluted with methanol (0.5 mL). The reaction mixture was purified by prep LC-MS with a gradient from 5% MeCN/water to 95% MeCN/water run over 14 mins. Pure fractions were evaporated to give 2-(1-(1-(2-chloro-4-fluorophenyl-sulfonyl)piperidin-4-yl)-2-oxo-1H-imidazo[4,5-c]pyridin-3(2H)-yl)acetic acid 140. LC-MS (+ESI) m/z=469 [M+H]⁺.

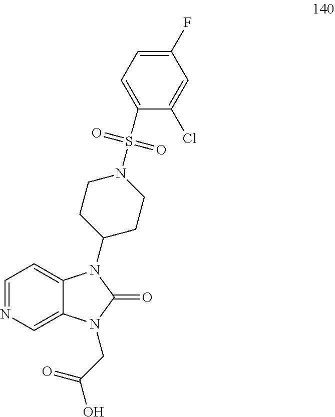

Compounds 141-144 were prepared according to the procedure described for the synthesis of compound 140 by the reaction of intermediate 139 with 2-chlorobenzene-sulfonyl chloride, 2,4-dichlorobenzenesulfonyl chloride, 2-methylsulfonylbenzene-sulfonyl chloride and 2-methyl-4-fluorobenzenesulfonyl chloride, respectively.
2-(1-(1-(2-chlorophenylsulfonyl)piperidin-4-yl)-2-oxo-1H-imidazo[4,5-c]pyridin-3(2H)-yl)acetic acid (141). LC-MS (+ESI) m/z=451 [M+H]⁺.
2-(1-(1-(2,4-dichlorophenylsulfonyl)piperidin-4-yl)-2-oxo-1H-imidazo[4,5-c]pyridin-3(2H)-yl)acetic acid (142). LC-MS (+ESI) m/z=485 [M+H]⁺.
2-(1-(1-(2-(methylsulfonyl)phenylsulfonyl)piperidin-4-yl)-2-oxo-1H-imidazo[4,5-c]pyridin-3(2H)-yl)acetic acid (143). LC-MS (+ESI) m/z=495 [M+H]⁺.
2-(1-(1-(4-fluoro-2-methylphenylsulfonyl)piperidin-4-yl)-2-oxo-1H-imidazo[4,5-c]pyridin-3(2H)-yl)acetic acid (144). LC-MS (+ESI) m/z=449 [M+H]⁺.

The synthesis of compound 149 and 150 were described in Scheme 9. Reaction of compound 1b with glycine ethyl ester under conventional or microwave heating conditions gave intermediate 145, which was hydrogenated with palladium on carbon with hydrogen to give intermediate 146. Reductive amination of intermediate 146 with 1-Boc-4-piperidone provided intermediate 147. Ring closure with phosgene followed by treatment with TFA give intermediate 148. Compounds 149 and 150 were obtained by sulfonylation with aryl sulfonyl chlorides followed by saponification with LiOH.

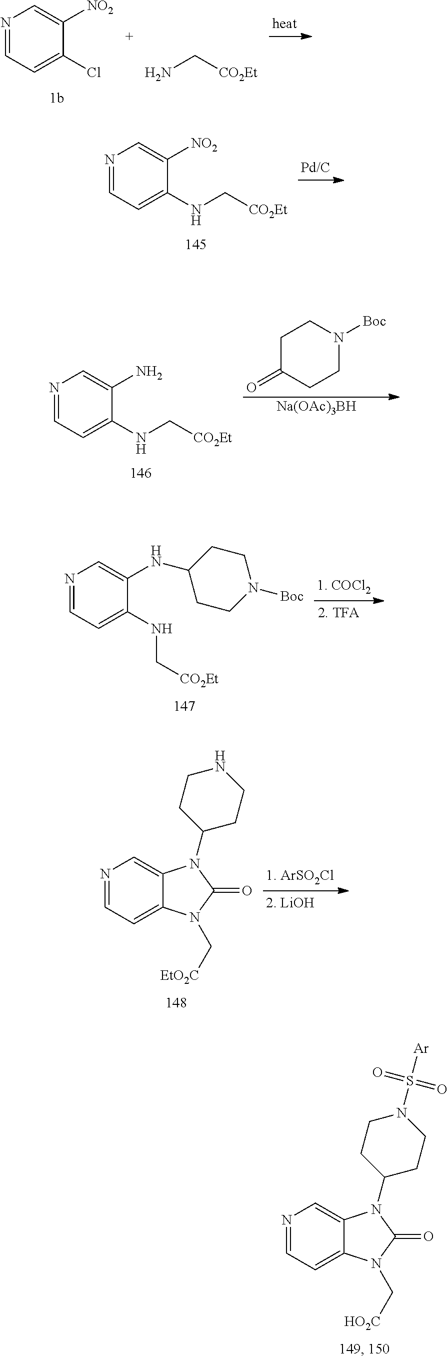

### Example 4

### Preparation of 2-(3-(1-(2-(methylsulfonyl)phenylsulfonyl) piperidin-4-yl)-2-oxo-2,3-dihydro-1H-imidazo[4,5-c]pyridin-1-yl)acetic acid (149)

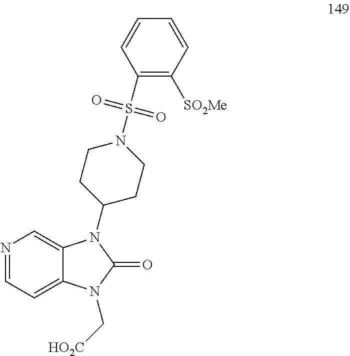

### Step 1: Preparation of ethyl 2-(3-nitropyridin-4-ylamino)acetate 145

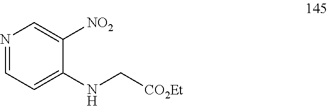

A mixture of 4-chloro-3-nitropyridine (1 g, 6.31 mmol) and ethyl 2-aminoacetate (0.976 g, 9.46 mmol) in Acetonitrile (10 mL) with DIEA (1.102 mL, 6.31 mmol) was heated at 150° C. for 30 min under microwave irradiation. The acetonitrile was removed under vacuum and the residue was brought up in EtOAc and washed with saturated NaHCO₃. The organic layer was separated, dried over Na₂SO₄ and concentrated to give ethyl 2-(3-nitropyridin-4-ylamino)acetate 145 in quantitative yield. LCMS (+ESI) m/z=226 [M+H]⁺.

### Step 2: Preparation of tert-butyl 4-(4-(2-ethoxy-2-oxoethylamino)pyridin-3-ylamino)piperidine-1-carboxylate 147

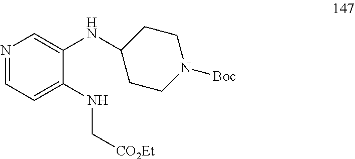

A mixture of ethyl 2-(3-nitropyridin-4-ylamino)acetate 145 (0.6 g, 2.66 mmol) and Pd/C (0.213 g, 1.998 mmol) in MeOH (10 mL) was hydrogenated under H₂ (40 psi) with a Parr shaker for 1 h. The reaction mixture was filtered through celite and concentrated under vacuum to give ethyl 2-(3-aminopyridin-4-ylamino)acetate 146 (0.52 g), which was stirred with tert-butyl 4-oxopiperidine-1-carboxylate (0.795 g, 3.99 mmol), formic acid (0.2 ml, 5.21 mmol) and sodium triacetoxyborohydride (0.846 g, 3.99 mmol) in DCE (5 mL) for 6 h. The reaction was quenched with 1N NaOH and the organic alyer was separated, dried over Na₂SO₄, and concentrated to give tert-butyl 4-(4-(2-ethoxy-2-oxoethylamino)pyridin-3-ylamino)piperidine-1-carboxylate 147 (0.83 g). LCMS (+ESI) m/z=379 [M+H]⁺.

### Step 3: Preparation of ethyl 2-(2-oxo-3-(piperidin-4-yl)-2,3-dihydro-1H-imidazo[4,5-c]pyridin-1-yl)acetate 148

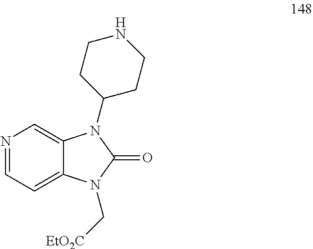

To a solution of tert-butyl 4-(4-(2-ethoxy-2-oxoethylamino)pyridin-3-ylamino)piperidine-1-carboxylate 147 (0.83 g, 2.193 mmol) and DIEA (0.77 mL) in DCM (5 mL) was added phosgene (20% in toluene, 1.3 mL). After stirring at r.t for 1 h, the reaction was quenched with saturated sodium bicarbonate. The organic phase was separated and dried over sodium sulfate. The crude product was purified by column chromatography to give a Boc protected intermediate, which was stirred in TFA/DCM at room teperature for 1 h. Evaporation of TFA/DCM gives ethyl 2-(2-oxo-3-(piperidin-4-yl)-2,3-dihydro-1H-imidazo[4,5-c]pyridin-1-yl)acetate 148, which was used as is in the next step. LCMS (+ESI) m/z=305 [M+H]⁺.

### Step 4: Preparation of 2-(3-(1-(2-(methylsulfonyl)phenylsulfonyl)piperidin-4-yl)-2-oxo-2,3-dihydro-1H-imidazo[4,5-c]pyridin-1-yl)acetic acid (149)

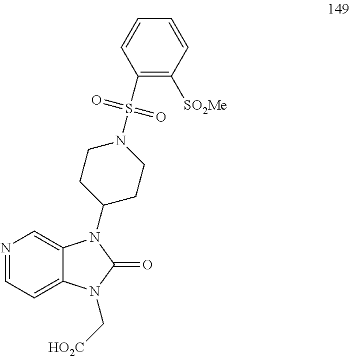

To a solution of ethyl 2-(2-oxo-3-(piperidin-4-yl)-2,3-dihydro-1H-imidazo[4,5-c]pyridin-1-yl)acetate 148 (20 mg, 0.066 mmol) and DIEA (23 uL) in DCM (1 mL) was added 2-(methylsulfonyl)benzene-1-sulfonyl chloride (0.020 g, 0.079 mmol). The reaction mixture was stirred at room teperature for 1 hr and then washed with saturated NaHCO₃. The organic layer was separated, dried over Na₂SO₄, filtered and concentrated. The residue was brought up in Methanol (0.5 mL) and a solution of lithium hydroxide (7.87 mg, 0.329 mmol) in water (0.5 mL) was added. The reactions were stirred at room teperature for 2 hr. The crude reaction mixture was purified by preparative LC-MS to give 2-(3-(1-(2-(methylsulfonyl)phenylsulfonyl)piperidin-4-yl)-2-oxo-2,3-dihydro-1H-imidazo[4,5-c]pyridin-1-yl)acetic acid. LCMS (+ESI) m/z=495 [M+H]⁺.

### Example 5

### Preparation of 2-(3-(1-(2-chloro-4-fluorophenylsulfonyl)piperidin-4-yl)-2-oxo-2,3-dihydro-1H-imidazo[4,5-c]pyridin-1-yl)acetic acid (150)

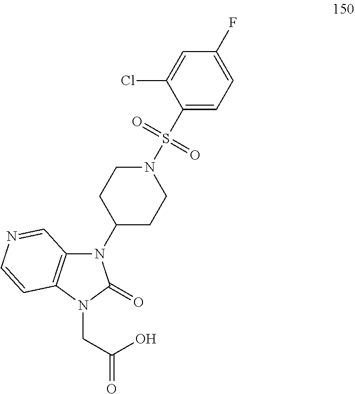

Compound 150 was synthesized by following the procedure described for the synthesis of compound 149 by replacing 2-(methylsulfonyl)benzene-1-sulfonyl chloride with 2-chloro-4-fluorobenzenesulfonyl chloride. LCMS (+ESI) m/z=469 [M+H]⁺.

### Example 6

### Further Examples of the compounds of formula I

Those of skill in the art will recognize that the following further examples of the compounds having the structure of formula I can be prepared according to the schemes and synthetic methods disclosed herein:

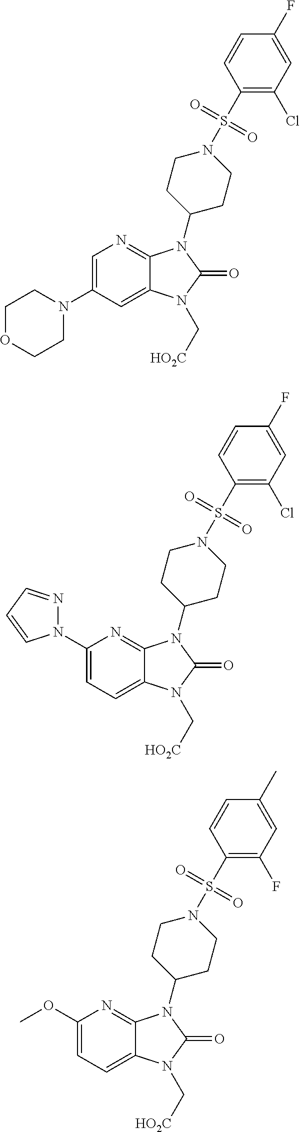

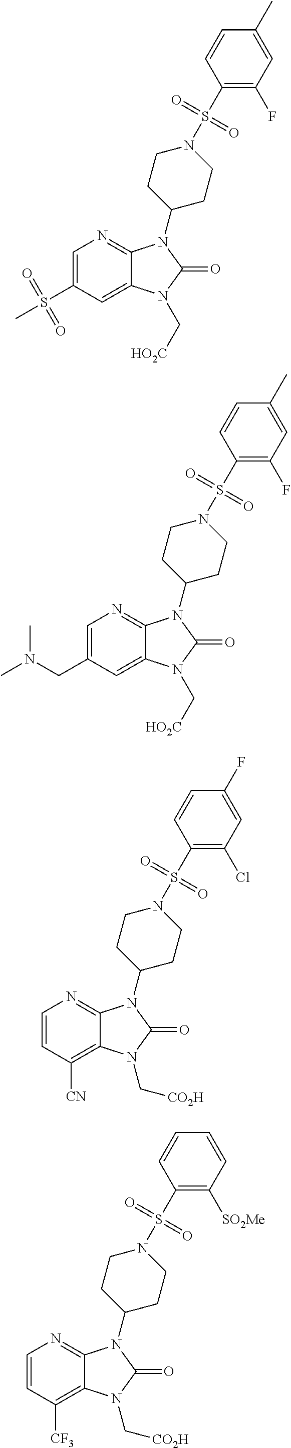

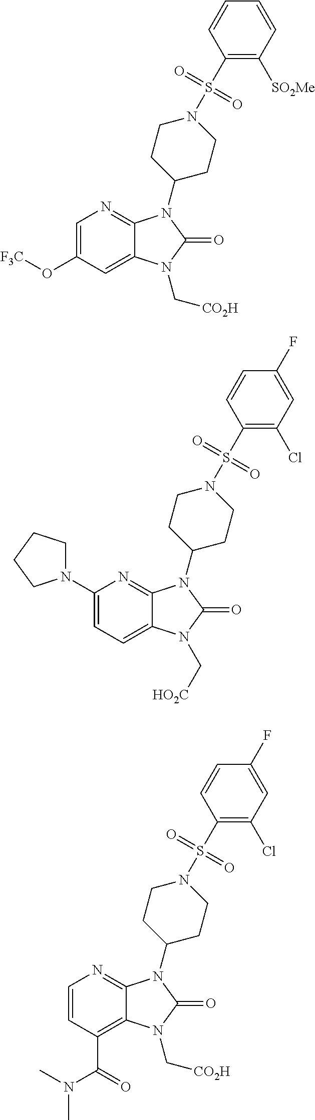

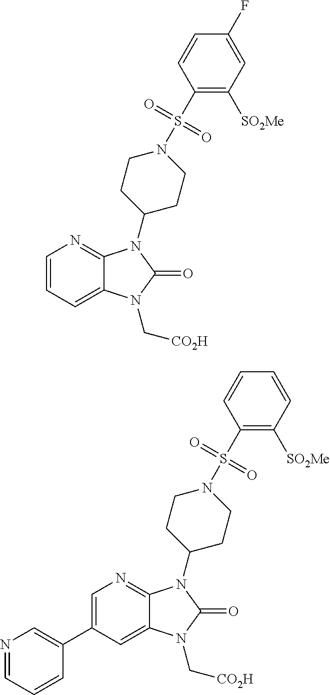

### Example 7

### Prostaglandin D2 Binding Inhibition Assay

The ability of compounds to inhibit binding of prostaglandin D2 (PGD₂) to the human DP₂ receptor was measured using a scintillation proximity assay (SPA). Chinese Hamster Ovary FLP-In cell lines (CHO-K1 FLP-In, Invitrogen, catalog no. R758-07) were stably transfected with the human DP2 gene (Genebank: AY507142). Cell lines were grown in suspension in EX-CELL 302 CHO Serum-free medium (Sigma, catalog no. 14324C) supplemented with 1% Fetal Bovine Serum (Invitrogen no. 10099-141), 2 mM L-glutamine (Invitrogen, catalog no. 25030-081) and 0.5 mg/ml Hygromycin B (Invitrogen, catalog no. 10687-010). Cells were collected by centrifugation in a Beckman Coulter Allegra X-12R centrifuge at 150×g for 5 minutes at room temperature.

Membranes from transfected cells were isolated for binding assays. Cell pellets containing approximately 2×10⁹ cells were resuspended in 35 ml of cold membrane homogenization buffer (10 mM Tris (Sigma, catalog no. T2663), 1 mM EDTA (Fluka BioChemika no. 03690), and 70 μl of protease inhibitor cocktail (Sigma, catalog no. P8340)). Cells were homogenized with 30 strokes of a dounce homogenizer (Kontes, catalog no. 885303-0040), and the solution centrifuged at 200×g for 10 minutes. The supernatant was transferred to a 50 ml Oakridge tube (Beckman, catalog no. 357000) and centrifuged in a Beckman Coulter Avanti J-26×PI centrifuge with a JA30.50 rotor at 81,000×g for 1 hour at 15° C. The pellet was resuspended in membrane storage buffer (50 mM Tris (Sigma, catalog no. T2663), 2.5 mM EDTA (Fluka BioChemika no. 03690), 5 mM magnesium chloride (Sigma, catalog no. M1028), 100 mg/ml sucrose (Sigma, catalog no. 50389) and a 1:500 dilution of protease inhibitor cocktail (Sigma, catalog no. P8340)) and the total protein was quantitated using a BCA protein measurement kit (Pierce, catalog no. 23225). Aliquots were frozen at −80° C. until used. Compounds were tested for their ability to inhibit PGD₂ binding to the purified membranes. 5 μg of the purified membranes, 0.25 mg of wheat germ agglutinin PVT SPA beads (GE Healthcare, catalog no. RPNQ0060) and 5.5 nM of ³H PGD₂ (PerkinElmer, catalog no. NET-616) were mixed in assay buffer ((50 mM Tris (Sigma, catalog no. T2663), 10 mM magnesium chloride (Sigma, catalog no. M1028), 100 mM sodium chloride (Sigma, catalog no. S6546) and 0.1% bovine serum albumin (Sigma, catalog no. A2153)). Using a Multidrop bulk dispenser, 75 μl of the assay mixture was dispensed in Optiplates (PerkinElmer, catalog no. 6007299) containing the compounds to be tested. After a 60 minute incubation period at room temperature, the plates were read on a PerkinElmer Topcount NXT HTS, and individual wells were quantitated as counts per minute (CPM). Data were expressed as a percent of the difference between the maximum and minimum responses. IC₅₀ values (the concentration of compound producing 50% of the maximal response) were determined using a two-parameter non-linear regression algorithm analyzed using CambridgeSoft Bioassay software. Table 2 gives the range of IC₅₀ values for claimed compounds in the binding assay.

TABLE 2
IC₅₀ Ranges for Inhibition of PGD2 Binding to Human DP2

| IC₅₀ | | COMPOUND NO. |
|---|---|---|
| <1 | μM | 30, 31, 36, 49, 51, 58, 63, 64, 67, 70, 74, 82, 83, 85, 86, |
| | | 87, 91, 93, 94, 96, 98, 100, 101, 102, 103, 111, 113, 114, |
| | | 115, 116 |
| 1-10 | μM | 32, 33, 34, 35, 37, 38, 39, 40, 41, 42, 44, 45, 46, 48, 50, |
| | | 52, 53, 55, 57, 59, 60, 61, 62, 65, 66, 69, 71, 72, 76, 77, |
| | | 78, 79, 80, 81, 84, 88, 89, 92, 95, 97, 99, 104, 105, 106, |
| | | 107, 108, 109, 110, 112, 124, 127, 149 |
| 10-30 | μM* | 43, 47, 56, 68, 73, 125, 126 |

*The upper limit of IC₅₀ of compounds determinable by the assay method was 30 μM. The IC₅₀ values for compounds 54, 75, 90, 117-123, 132-135, 140-144 and 150 were above the determinable range.

### Example 8

### Measurement of Inhibition of Intracellular cAMP Induction

The ability of compounds to act as antagonists or agonists at the human DP₂ receptor was determined by measuring changes in intracellular cAMP levels using a LANCE cAMP detection kit (PerkinElmer, catalog no. AD0264) utilizing time-resolved fluorescence resonance energy transfer (TR-FRET).

Human DP2 cells were grown as described above. Cells were diluted to 2×10⁵ cells/mL, and pre-incubated with 0.5 mM 3-isobutyl-3-methylzanthanine or IBMX (Sigma, catalog no. I7018). Antibody was added to all the cells as per the manufacturer's instructions. For antagonist experiments, cells were then pre-stimulated for 15 minutes with 4 μM of the water soluble forskolin derivative NKH-477 (Tocris, catalog no. 1603) to increase intracellular cAMP levels. An aliquot of cells was removed for use in uninduced controls. Using a Multidrop bulk dispenser, 2×10³ cells per well were then added to Proxiplates containing test compounds and 30 nM PGD2. Plates were incubated for sixty minutes, and the response stopped by addition of a detection mix according to the manufacturer's instructions. After a three hour equilibration, plates were read on an Envision multi-mode detector (PerkinElmer). TR-FRET was measured using a 330-380 nm excitation filter, 615 and 665 nm emission filters, and a 380 nm dichroic mirror set at a Z-height of 11 mm.

The cAMP concentration in each well was back-calculated from a cAMP standard curve run concurrently with each assay. Cyclic AMP responses were expressed as a percent of the difference between the maximum and minimum responses, determined from the induced and uninduced controls, respectively. EC₅₀ values (the concentration of compound producing 50% of the maximal response) were determined using a four-parameter non-linear regression algorithm (CambridgeSoft Bioassay).

Compound 63: 2-(3-(1-(2-(methylsulfonyl)phenylsulfonyl)piperidin-4-yl)-2-oxo-2,3-dihydro-1H-imidazo[4,5-b]pyridin-1-yl)acetic acid acted as an antagonist of intracellular cAMP induction, with an EC₅₀ of less than 1 μM as determined by the above-described method.

### Example 9

### In vivo Assay Methods

The compounds are tested in vivo in a rodent model (particularly a rat or mouse model) based on detection of extravasation of Evans Blue dye mediated by local injection of PGD2 intradermally. The effect of administration of a compound of the invention before PGD2 administration is then determined. A reduction in extravasation and/or edema due to compound administration indicates antagonism of PGD2-mediated inflammation.

Alternatively, compounds are tested in vivo in a rodent model (particularly a rat or mouse model) based on monitoring of paw withdrawal thresholds upon stimulation with a von Frey filaments following sensitization by injection of the paw with PGD2. A reduction in sensitization (i.e. allodynia) due to compound administration indicates antagonism of the PGD2-mediated allodynia.

The texts of the references cited in this specification are incorporated herein by reference in their entireties. In the event that any definition or description contained in one or more of these references is in conflict with the corresponding definition or description in this specification, then the definition or description disclosed herein is intended. The examples provided herein are for illustration purposes, and are not intended to be taken as limitations to the scope of the invention, the full breadth of which will be readily recognized by those of skill in the art.

We claim:

1. A compound having the structure: 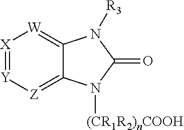
or a stereoisomer, prodrug, pharmaceutically acceptable salt, hydrate, salt hydrate, acid salt hydrate, or polymorph thereof;
wherein one member of the group consisting of W, X, Y and Z is N and the remaining three members of the group consisting of W, X, Y and Z are each independently CR₄;
R₁ and R₂ are each independently H or C₁-C₆ alkyl;
R₃ is 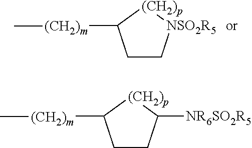
each instance of R₄ is independently selected from the group consisting of H, OH, halo, NO₂, CN, C₁-C₆ alkylsulfonyl, C₁-C₆ alkyl, C₁-C₆ haloalkyl, C₁-C₆ alkoxy, C₁-C₆ haloalkoxy, C₁-C₆ aminoalkyl, C₁-C₆ alkylamino, C₁-C₆ alkylcarbamoyl, 6- to 10-membered aryl and 4- to 10-membered heterocyclyl;
R₅ is: (i) 6- to 10-membered aryl optionally substituted with from 1 to 4 groups independently selected from the group consisting of OH, halo, NO₂, CN, C₁-C₆ alkylsulfonyl, C₁-C₆ alkyl, C₁-C₆ haloalkyl, C₁-C₆ alkoxy, C₁-C₆ haloalkoxy, C₁-C₆ aminoalkyl, C₁-C₆ alkylamino, C₁-C₆ alkylcarbamoyl, heteroaryl, phenyl and phenoxy;
(ii) C₁-C₁₀ alkyl or C₃-Cₛ cycloalkyl, each optionally substituted with from 1 to 4 groups independently selected from the group consisting of halo, OH, NO₂, CN, C₁-C₆ alkyl, C₁-C₆ haloalkyl, C₁-C₆ alkoxy, C₁-C₆ haloalkoxy, C₁-C₆ alkylsulfonyl and phenyl; or
(iii) 4- to 10-membered heterocyclyl optionally substituted with from 1 to 4 groups independently selected from the group consisting of halo, OH, NO₂, CN, C₁-C₆ alkyl, C₁-C₆ haloalkyl, C₁-C₆ alkoxy, C₁-C₆ haloalkoxy and C₁-C₆ alkylsulfonyl;
R₆ is H or C₁-C₄ alkyl; and
wherein m is 0, 1, 2 or 3; n is 1, 2, 3 or 4; and p is 1, 2 or 3.

2. The compound according to claim 1, wherein R₁ and R₂ are each independently H, Me or Et, and n is an integer from 1 to 3.

3. The compound according to claim 2, wherein R₁ and R₂ are each H and n is 1.

4. The compound according to claim 3, wherein p is 1 or 2.

5. The compound according to claim 4, wherein m is 0 and p is 2.

6. The compound according to claim 4, wherein m is 0 and p is 1.

7. The compound according to claim 3, wherein at least one instance of R₄ is H.

8. The compound according to claim 7, wherein at least two instances of R₄ are H.

9. The compound according to claim 3, wherein R₅ is 6- to 10-membered aryl optionally substituted with from 1 to 4 groups independently selected from the group consisting of OH, halo, NO₂, CN, C₁-C₆ alkylsulfonyl, C₁-C₆ alkyl, C₁-C₆ haloalkyl, C₁-C₆ alkoxy, C₁-C₆ haloalkoxy, C₁-C₆ aminoalkyl, C₁-C₆ alkylamino, C₁-C₆ alkyl-carbamoyl, phenyl and phenoxy.

10. The compound according to claim 9, wherein the optionally substituted aryl of R₅ is a 6-membered aryl.

11. The compound according to claim 10, wherein the 6-membered aryl is optionally substituted with from 1 to 4 groups independently selected from the group consisting of halo, CN, C₁-C₆ alkylsulfonyl, C₁-C₆ alkyl, C₁-C₆ haloalkyl and C₁-C₆ alkoxy.

12. The compound according to claim 3, wherein R₅ is C₁-C₁₀ alkyl or C₃-C₈ cycloalkyl, each optionally substituted with from 1 to 4 groups independently selected from the group consisting of halo, OH, NO₂, CN, C₁-C₆ alkyl, C₁-C₆ haloalkyl, C₁-C₆ alkoxy, C₁-C₆ haloalkoxy, C₁-C₆ alkylsulfonyl and phenyl.

13. The compound according to claim 12, wherein R₅ is C₁-C₁₀ alkyl optionally substituted with from 1 to 4 groups independently selected from the group consisting of halo, OH, NO₂, CN, C₁-C₆ alkoxy and C₁-C₆ haloalkoxy.

14. The compound according to claim 13, wherein the optional substituents are independently selected from the group consisting of halo and C₁-C₆ alkoxy.

15. The compound according to claim 3, wherein R₅ is 4- to 10-membered heterocyclyl optionally substituted with from 1 to 4 groups independently selected from the group consisting of halo, OH, NO₂, CN, C₁-C₆ alkyl, C₁-C₆ haloalkyl, C₁-C₆ alkoxy, C₁-C₆ haloalkoxy and C₁-C₆ alkylsulfonyl.

16. The compound according to claim 15, wherein the optionally substituted heterocyclyl of R₅ is a 5-, 6- or 7-membered heterocyclyl.

17. The compound according to claim 16, wherein the optional substituents of the heterocyclyl are independently selected from the group consisting of halo, C₁-C₆ alkyl, C₁-C₆ haloalkyl, C₁-C₆ alkoxy and C₁-C₆ alkylsulfonyl.

18. A pharmaceutical composition comprising a compound according to claim 1 and a pharmaceutically acceptable vehicle, diluent or excipient.

19. A method of prophylaxis or treatment of a disease or condition mediated by or addressable by DP2, the method comprising administering an effective amount of a compound according to claim 1 to a subject in need thereof.

20. The method of claim 19, wherein the disease or condition is selected from the group consisting of pain, inflammation and allergy.
